(19) United States
(12) Patent Application Publication
(10) Pub. No.: US 2021/0386888 A1
(43) Pub. Date: Dec. 16, 2021

(54) SANITIZATION SYSTEMS AND METHODS

(71) Applicant: OAK MOUNTAIN SOLUTIONS, LLC, Indian Springs, AL (US)

(72) Inventors: Robert Hugh Lee, Indian Springs, AL (US); Tyler Stone, Homewood, AL (US)

(21) Appl. No.: 17/347,170

(22) Filed: Jun. 14, 2021

Related U.S. Application Data
(60) Provisional application No. 63/113,693, filed on Nov. 13, 2020.
(60) Provisional application No. 63/038,707, filed on Jun. 12, 2020.

(51) Int. Cl. A61L 2/18; A61L 2/24

(52) U.S. Cl. CPC A61L 2/18; A61L 2202/15; A61L 2202/122; A61L 2/24

(57) ABSTRACT

A system for systematically disinfecting shopping carts and other items includes an arch through which shopping carts or other items may be passed and disinfected. For disinfecting, carts are pushed through a passageway defined by the arch while nozzles positioned in the arch are actuated to dispense a disinfecting fluid. A fine mist of disinfecting fluid envelopes all surfaces of the cart as it passes through the passageway, thereby sanitizing the entire surface area of the cart with a reduction or elimination of residue accumulation. When spraying is deactivated, at least one solenoid is controlled to depressurize the tubing connected to the nozzles.

### CROSS REFERENCE TO RELATED APPLICATION

This application claims priority to U.S. Provisional Application No. 63/038,707, entitled “Sanitization System and Device” and filed on Jun. 12, 2020, which is incorporated herein by reference. This application also claims priority to U.S. Provisional Application No. 63/113,693, entitled “Sanitization Systems and Methods” and filed on Nov. 13, 2020, which is incorporated herein by reference.

### RELATED ART

Today's society is highly mobile. Travel across large distances, such as from one country to another across multiple continents, sometimes in a single twenty-four hour period, is common. Infectious diseases follow humans wherever they go and our societal mobility, while enhancing business and prosperity, brings with it the spread of disease. While many diseases are spread with human-to-human contact or proximity, many diseases are also spread through germs acquired through contact surfaces. Consumers are rapidly becoming aware of this exposure threat.

Recently, consumers have watched as new infectious diseases have surfaced and quickly spread globally. The increasing awareness of the historical effects of past influenza epidemics, pandemic illness outbreaks, and the current incurability of many human viruses, the consuming public has become very aware of the potential for catching diseases from contact with germs. Consumers now look for and routinely take advantage of antibacterial and anti-viral procedures available to them in retail store locations. Further, retail stores are taking active measures to reduce the spread of germs to consumers visiting their premises.

One area of significant concern is grocery outlets. A grocery outlet not only exposes consumers to human spread contagions, but food borne illnesses that are spread by germs are encountered throughout the entire store. Also, consumers use various styles of shopping carts such as a standard wheeled cart or a small hand basket, and germs are spread to users through contact with the handles and surrounding structure of each cart or container. Shopping carts and hand baskets come into contact with more customers and more food products than any other area of a grocery store, and store customers often do not clean their hands before utilizing shopping carts or hand baskets. In addition, many customers do not wear protective gloves to shield themselves from exposure to the number of possible germs that may reside on the shopping cart or basket.

Grocery stores are aware of this concern by consumers. In response, the average grocery store may pressure wash carts and hand baskets with water (not killing any germs) periodically, say every few months or so. Because retail storeowners have needed to make some effort to address the concentration of germs on shopping carts and hand baskets, some stores have begun providing alcohol-based wipes and hand sanitizing liquid adjacent to the carts and baskets so that customers may attempt to address the germ problem themselves. The consumer can apply the hand sanitizer to their hands, before and after shopping, and they can use the alcohol wipes to disinfect shopping cart or basket surfaces. Unfortunately, alcohol wipes address only a small percentage of the contact potential of a cart surface, and there is no quality control as to how well or poorly consumers might attempt to disinfect their hands or a cart surface. Further, there is a need in retail stores, entertainment and sports facilities, factories where different shifts of workers use the same tools, conveyor systems, airports, medical facilities and many other places that need an efficient way to sanitize physical objects with which users come into contact.

Hence, what is needed are systems and methods to efficiently and systematically sanitize grocery carts and baskets while avoiding inconvenience to consumers so that the risk of exposing customers to germs and disease during their shopping experience is reduced.

### BRIEF DESCRIPTION OF THE DRAWINGS

The disclosure can be better understood with reference to the following drawings. The elements of the drawings are not necessarily to scale relative to each other, emphasis instead being placed upon clearly illustrating the principles of the disclosure. Furthermore, like reference numerals designate corresponding parts throughout the several views.

FIG. 1 a front perspective view of an embodiment of a shopping cart disinfecting system showing its exterior.

FIG. 2A is a front view of the shopping cart disinfecting system depicted by FIG. 1.

FIG. 2B is a rear view of the shopping cart disinfecting system depicted by FIG. 1.

FIG. 2C is a left side view of the shopping cart disinfecting system depicted by FIG. 1.

FIG. 2D is a right side view of the shopping cart disinfecting system depicted by FIG. 1 showing the positioning of a hand sprayer and external control elements.

FIG. 3 is a front perspective view of the shopping cart disinfecting system depicted by FIG. 1 with a front face of the system removed to show interior elements.

FIG. 4A is a perspective interior view of a left access door of the system depicted by FIG. 1 with the left access door in the open position.

FIG. 4B is a perspective interior view of an upper access door of the system depicted by FIG. 1 with the upper access door in the open position.

FIG. 4C is a magnified view of a spray nozzle tip in an access door of the system depicted by FIG. 1.

FIG. 5 is a front view of the shopping cart disinfecting system depicted by FIG. 1 showing exemplary dynamics of sanitizing a shopping cart.

FIG. 6 is a process flow diagram showing exemplary steps in disinfecting one or more shopping carts using a shopping cart disinfecting system, such as is depicted by FIG. 1.

FIG. 7 is a front perspective view of an embodiment of a shopping cart disinfecting system showing its exterior.

FIG. 8A is a front view of the shopping cart disinfecting system depicted by FIG. 7.

FIG. 8B is a rear view of the shopping cart disinfecting system depicted by FIG. 7.

FIG. 8C is a left side view of the shopping cart disinfecting system depicted by FIG. 7.

FIG. 8D is a right side view of the shopping cart disinfecting system depicted by FIG. 7 showing the positioning of a hand sprayer and external control elements.

FIG. 9 is a front perspective view of the shopping cart disinfecting system depicted by FIG. 7 with the front face removed to show interior elements.

FIG. 10A is a perspective interior view of a left access door of the system depicted by FIG. 7 with the left access door in the open position.

FIG. 10B is a perspective interior view of an upper access door of the system depicted by FIG. 7 with the upper access door in the open position.

FIG. 10C is a magnified view of a spray nozzle tip in an access door of the system depicted by FIG. 7.

FIG. 11 is a front view of the shopping cart disinfecting system depicted by FIG. 7 showing exemplary dynamics of sanitizing a shopping cart.

FIG. 12 is a process flow diagram showing exemplary steps in disinfecting one or more shopping carts using a shopping cart disinfecting system, such as is depicted by FIG. 7.

FIG. 13 is an interior view of the left side of the system depicted by FIG. 7 showing exemplary pumping system components mounted on a backpan.

FIG. 14 is a block diagram illustrating an exemplary embodiment of various components of a shopping cart disinfecting system, such as is depicted by FIG. 7.

FIG. 15 is a side view of components situated within a housing of a shopping cart disinfecting system, such as is depicted by FIG. 7.

FIG. 16 is a block diagram illustrating an exemplary embodiment of a solenoid used in a shopping cart disinfecting system, such as is depicted by FIG. 7.

FIG. 17 is a block diagram illustrating an exemplary embodiment of a solenoid used in a shopping cart disinfecting system, such as is depicted by FIG. 7.

FIG. 18 is a side view and a front view showing a shopping cart disinfecting system, such as is depicted by FIG. 7.

FIG. 19 is a side view and a front view showing a shopping cart disinfecting system, such as is depicted by FIG. 7.

FIG. 20 is a side view showing a shopping cart disinfecting system, such as is depicted by FIG. 7, during maintenance operations on the system.

FIG. 21 is a side view and a front view showing a support structure of a shopping cart disinfecting system, such as is depicted by FIG. 7.

FIG. 22 is a side view showing the support structure of FIG. 21.

FIG. 23 is a side view showing the support structure of FIG. 21 with a removable base attached to the support structure and with various components mounted on the removable base.

FIG. 24 is a perspective view showing the removable base of FIG. 23.

FIG. 25 is a front view and a side view showing the support structure of FIG. 21 positioned on a base plate along with another support structure for supporting the housing and handle of a shopping cart disinfecting system, such as is depicted by FIG. 7.

FIG. 26 is a side view showing the support structure and base plate of FIG. 25.

FIG. 27 is a top view and a front view showing the base plate of FIG. 25.

FIG. 28 is a front view of a support structure attached to a handle through a wall of a housing for a shopping cart disinfecting system, such as depicted by FIG. 7.

FIG. 29 is a perspective view of a nozzle of a shopping cart disinfecting system, such as is depicted by FIG. 7.

FIG. 30 is an exploded view of the nozzle depicted by FIG. 29.

FIG. 31 is a perspective view of a bracket and nozzle of a shopping cart disinfecting system, such as is depicted by FIG. 7.

FIG. 32 is a perspective view of the bracket and nozzle depicted by FIG. 31 with the nozzle connected to a swivel fitting.

FIG. 33 is a perspective view of the bracket, nozzle, and swivel fitting depicted by FIG. 32 mounted on an inner wall of a housing for a shopping cart disinfecting system, such as is depicted by FIG. 7.

FIG. 34 is a perspective view of an interior of a housing of a shopping cart disinfecting system, such as is depicted by FIG. 7, after the housing has been positioned on and secured to a base plate.

FIG. 35 is a perspective view of an exterior of the housing depicted by FIG. 34.

FIG. 36 is a perspective view of a front of the housing depicted by FIG. 7.

### DETAILED DESCRIPTION

The present disclosure generally pertains to systems and methods for sanitizing shopping carts and other items. In some embodiments, the system comprises an arch through which shopping carts or other items may be passed and disinfected. For disinfecting, carts are pushed through a passageway defined by the arch while nozzles positioned in the arch are actuated to dispense a disinfecting fluid, also sometimes referred to as a “sanitizing fluid.” A fine mist of disinfecting fluid envelopes all surfaces of the cart as it passes through the passageway, thereby sanitizing the entire surface area of the cart without accumulating residue.

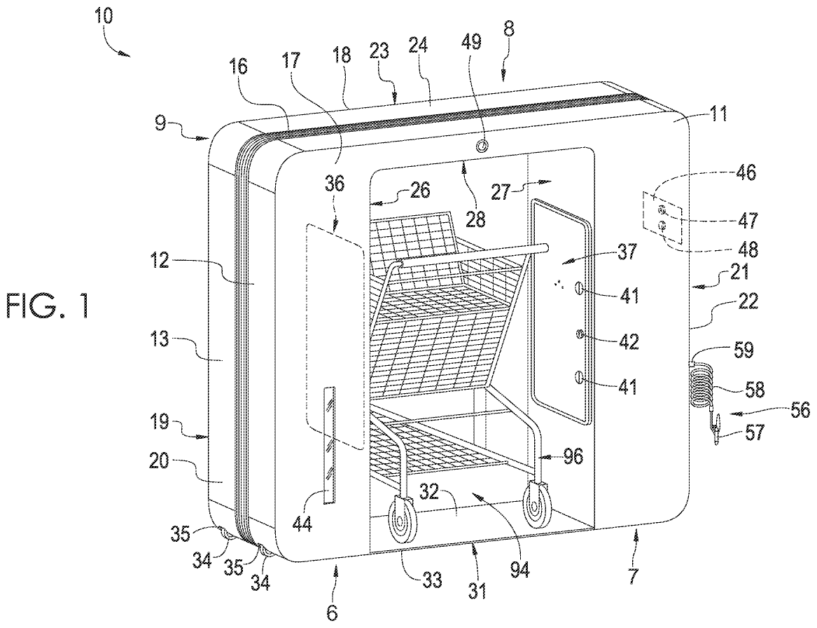

FIG. 1 shows a system 10 for sanitizing one or more shopping carts 96. The system 10 is comprises an arch 9 having a housing 11 that is hollow on the interior and formed into an inverted U-shape. The arch has a pair of legs 6, 7 that extend downward in a vertical direction, and the legs 6, 7 are joined by a top section 8 that extends horizontally as shown. The housing 11 may be composed of fiberglass or some other suitable material and has a front section 12 and rear 13 section joined together with a hard plastic molding 16. The joined sections form a front face or surface 17, a rear facing surface 18, left and right sides 19, 21 having left and right surfaces 20, 22, and a top or upper portion 23 with a top surface 24. The arch forms a bounded interior sanitization zone 94 through which one or more shopping carts or other items to be disinfected may pass. The sanitization zone 94 is defined by left and right inner surfaces 26, 27 of the housing 11, and an inner surface 28 of the upper portion 23 of the housing 11. Each of these inner surfaces 26-28 includes an access door 36, 37, and 38, respectively with hand laches 41, and the side access doors 36, 37 includes key access locks 42 as shown and as will be further described below. A level indicator window 44 is formed in front surface 17 to allow viewing of fluid levels in a disinfectant storage container inside housing 11. The underside of the housing 11 includes a base plate 31, having an upper surface 32 and an under surface 33.

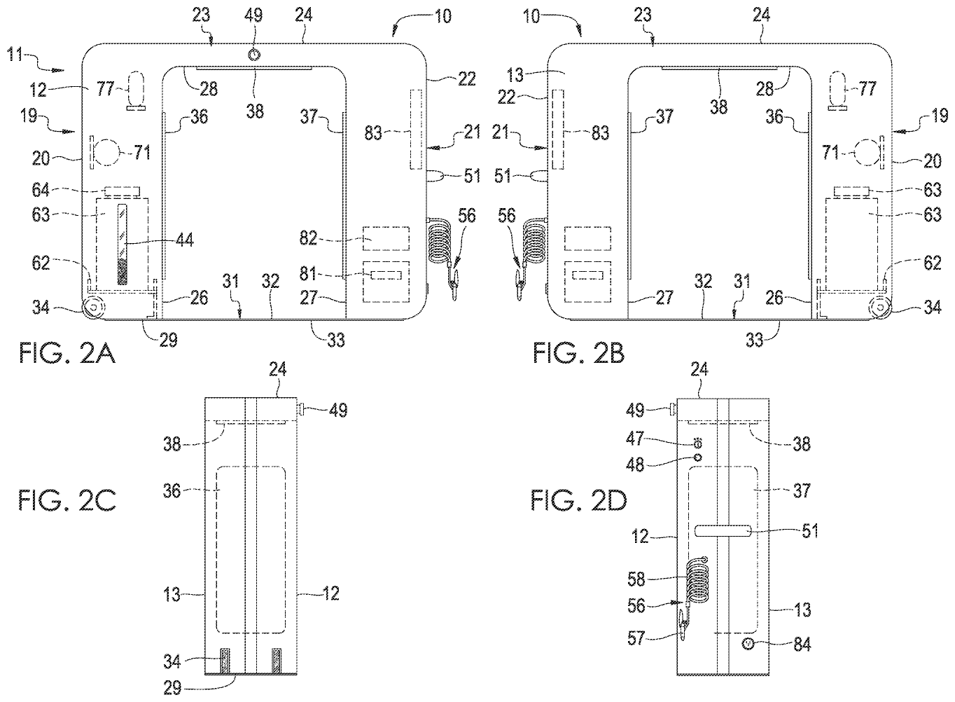

Referring now to FIGS. 2A-2D, it may be seen in FIG. 2A that the system 10 has a front actuation switch 49 for activating the system 10, and specifically initiating a spraying cycle. A pair of wheels 34 are affixed to a frame 62 within the left side 19 and extend through two slots 35 (FIG. 1) formed in the housing 11 at the juncture of the lower edge of left side surface 20 and bottom surface 29 of housing left side 19. A countervailing handle 51 is affixed to right side surface 22 and upon the lifting of right side 21, the wheels 34 will contact any ground surface supporting the system 10 and allow it to be easily moved and rotated as desired to reposition the system 10. The wheels 34 are positioned on frame 62 such that they do not contact the supporting ground until tilted by the lifting of opposite side end 21. Hence, the system 10 is very stable when resting in a horizontal position.

On the interior of left side 19, a fluid tank 63 rests on frame 62, which is sized to hold the tank 63 securely. A window 44 allows for viewing of a graduated measuring scale formed in the front side of tank 63, which has a translucent exterior so that a user can see the amount of fluid left in tank 63.

On the interior of the right side 21, the system 10 includes a battery 81 and charger 82 or other type of power supply for providing electrical power to electrical components of the system 10. A control panel 83 may be connected to the charger 82 and battery 81 to provide power to other electrical components of the system 10 and to monitory and control the distribution of the power throughout the system 10 to power, for example, pump 71. The charger 82 and battery 81 may be affixed directly to the one or more walls of the housing 11 and need no separate support frame. The charger 82 may be connected to a power outlet (not shown) to allow for a plug and wire to connect system 10 with a standard household electrical outlet to the charge battery 81. An access panel 46 has one or more control elements, such as a key switch 47 and a stop indicator light 48, that are affixed to and extending through the right side surface 22. The stop indicator light 48 may be used to advise the operator of warning conditions in the system 10, such as for example when fluid levels have dropped below a certain threshold inside the tank 63. The key switch 47 may be a 3-position switch providing for an off position and two operating modes for system 10, though other numbers of key positions and modes are possible in other embodiments.

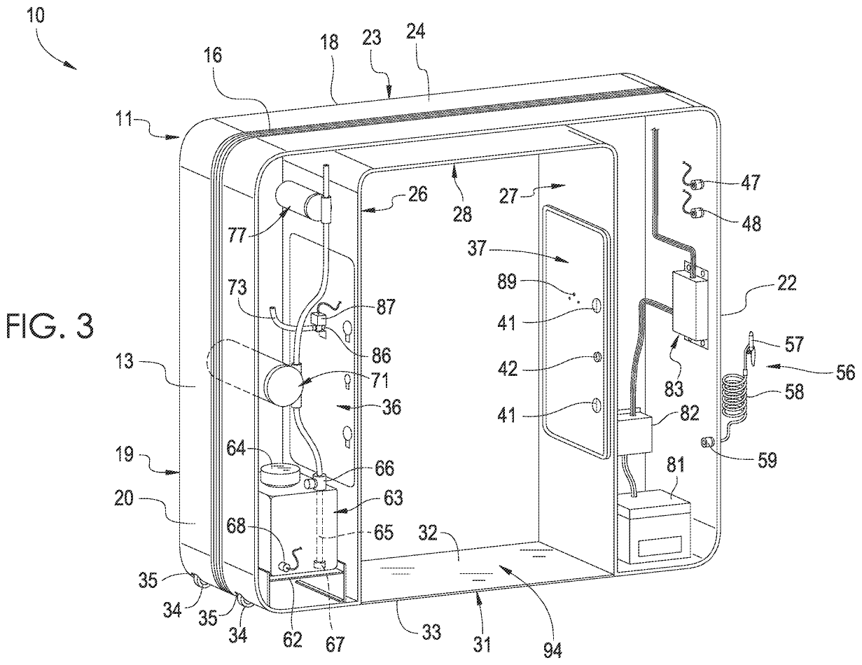

FIG. 3 shows a cut-away view of the system 10 showing various internal components. The tank 63 may hold approximately 7 gallons of sanitizing fluid, though other tank volumes are possible in other embodiments. Fluid may be added to the tank 63 by opening access door 36 and removing screw top lid 64. Filters 66, 67 may be fixed to the top and bottom of tank fluid access riser tube 65 for keeping debris that enter into the fluid tank 63 from being sucked into the internal fluid lines of the system 10. In some embodiments, a float sensor 68 may be positioned in the bottom of the tank 63 and provide a signal to controller 83 when the tank 63 is empty, and in response to such signal, the controller 83 may force the suspension of pump operation until fluid is replaced in the tank 63.

A network of fluid tubes travels throughout the inside of the housing 11 to connect the tank 63, pump 71, accumulator 77 and nozzles 86. The tubing 73 is resilient enough to hold sanitizing fluid under pressure, such as at least 100 psi pressure, for an extended period of time and includes connectors (not shown) for connecting the tubing 73 to the various internal components in the system 10. Nylon type tubes provide sufficient resiliency and durability to satisfy this requirement. Pump 71 can be a 12 Volt diaphragm type pump, such as manufactured by Shurflo Pump Manufacturing Co. of Cypress, Calif., under the name 8000 Series Diaphragm Pump, though other types of pumps may be used. The pump 71 can produce at least 100 psi and 1.15 gallons per minute through lines 73, as may be desired, but typical flow rates may be much less than 100 psi since spray nozzles 86 generally would not allow fluid to drain quickly enough for pump 71 to meet its flow rate upper limit. Tank accumulator 77, such as a model 181-201 type also available from Shurflo, keeps lines 73 under pressure within a typical range of about 65-100 psi to each nozzle, but it also reduces the actuation load on pump 71 by allowing it to cycle on and off less frequently while still managing a maximum pressure of about 100 psi and a minimum pressure of about 65 psi, as directed by internal logic in the pump's electronics. In other embodiments, other pressure ranges are possible.

The controller 83 may be implemented in hardware or any combination of hardware, software, and firmware. In some embodiments, the controller 83 comprises a processor (e.g., a microprocessor) programmed with instructions for performing the functions of the controller 83, as described in more detail herein. In other embodiments, other types of controllers 83, including field programmable gate arrays (FPGAs) and application-specific integrated circuits (ASICs) may be used.

The controller 83 is configured to control and provide power to operate the pump 71, which maintains the pressure in tubing 73 as described above. The controller 83 also is connected with wiring 76 to each solenoid 87 at each nozzle 86, actuation switch 49, key control switch 47, as well as providing power to the pump 71. The battery 81 may provide power to the controller 83, pump 71 (via controller 83 or otherwise), and all control lines for switches and solenoids. The charger 82 may be connected to an external plug 84 to provide 12 Volt (or other voltage) power to the battery 81 for its charging. The controller 83 may be reprogrammed or otherwise reconfigured as may be desired to accommodate the various needs of a shopping venue and its employees.

Various types of sanitizing fluid may be used in system 10. As an example, a diluted solution of “66” manufactured by Staples Contract & Commercial (Product Item No. STP660001-B) works satisfactorily as a solution to be held by the tank 63, but other types of sanitizing fluids may be used in other embodiments. The standard dilutions instructions that come with 66 and associated with this product will achieve satisfactory sanitization results when carts are sanitized in accordance with the herein provided methods and systems.

A hand sprayer 56 for spraying sanitizing fluid from the fluid tank 63 is positioned on the exterior of the right side 21 of the housing 11 and is connected by a coiled tube 58 to tubing 73 via connector 59 that is mounted on an inner wall of the right side 21. The sprayer 56 may be fluidly connected to the accumulator 77 via fluid lines 73 such that between about 65-100 psi fluid pressure is available to the hand sprayer 56 at all times or selected times as may be desired. When not in use, the handle 57 of sprayer 56 may be loosely hooked onto the mobility handle 51 or otherwise positioned as may be desired. In other embodiments, the hand sprayer 56 can be located on the front of the housing 11 such that it is in close positional relationship with the fluid tank 63. In other embodiments, yet other locations of the hand sprayer 56

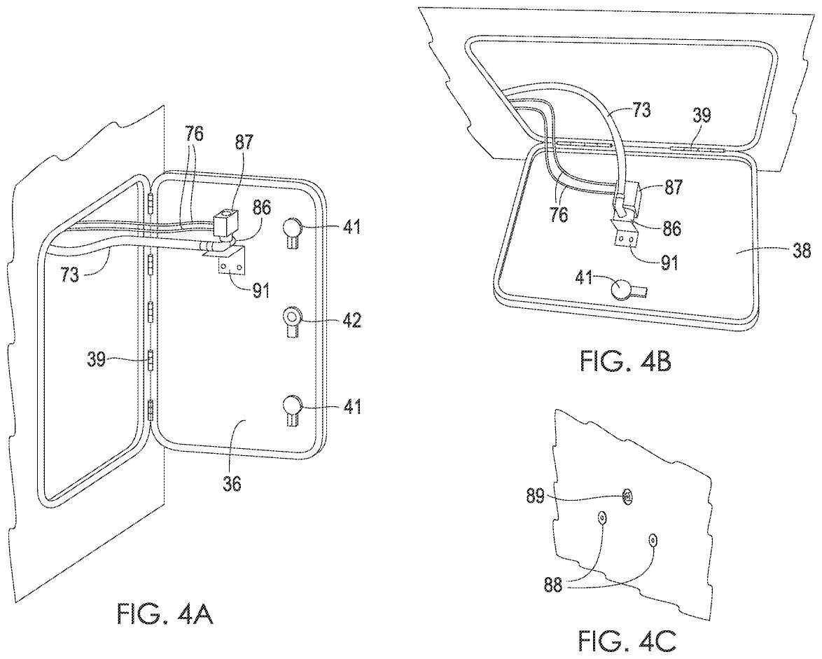

A closer view of the access doors of the system 10 and their elements may be seen in FIGS. 4A-4C. FIGS. 4A and 4B show the left access door 36 and the upper access door 38 in their open positions, each held to interior surface of the housing 11 with hinges 39. The right access door 37 is a mirror image of the left access door 36. Each access door 36, 37, 38, includes a lock 42, and rotating plastic latches 41 to secure each access door in a closed position. Nozzles 86 are affixed on the interior of each access door and positioned with nozzle tips 89 penetrating through the composite plastic of each doors' surface (see FIG. 4C). Solenoids 87 are positioned adjacent to each nozzle 86 to control the flow of sanitizing fluid to each nozzle 86. A respective tube 73 connects each nozzle 86 to the accumulator tank 77 and electrical wiring 76 operatively connects each solenoid 87 to the controller 83. For each nozzle 87, a corresponding small bracket 91 is riveted 88 (See FIG. 4C) to the surface of each door and holds the nozzle and its respective solenoid 87 in place. By placing a spray nozzle 86 in each access door, the cart sanitization zone 94 is surrounded by three intersecting spray patterns such that each cart passing through the zone 94 will have close to 100 percent of its surface areas covered by sanitizing fluid.

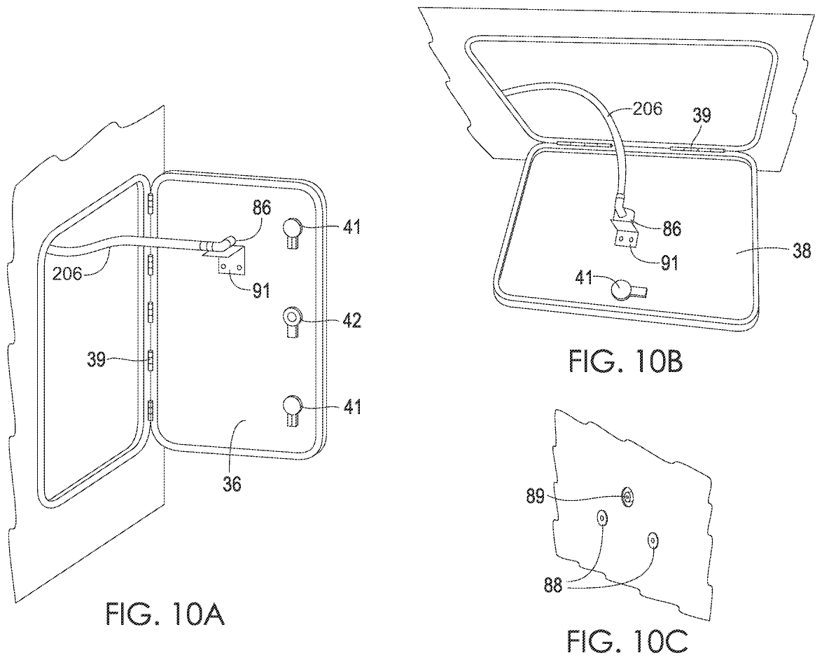

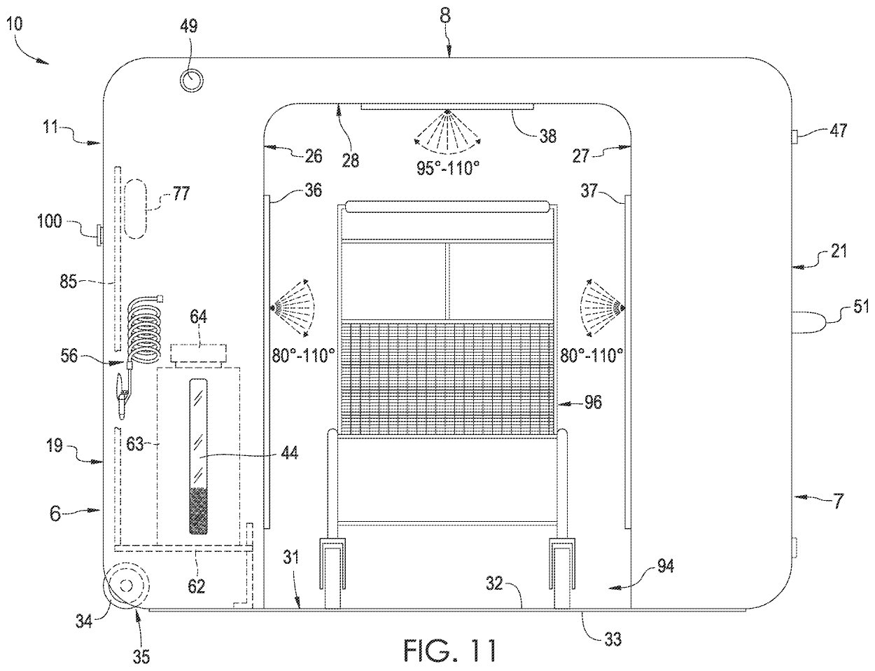

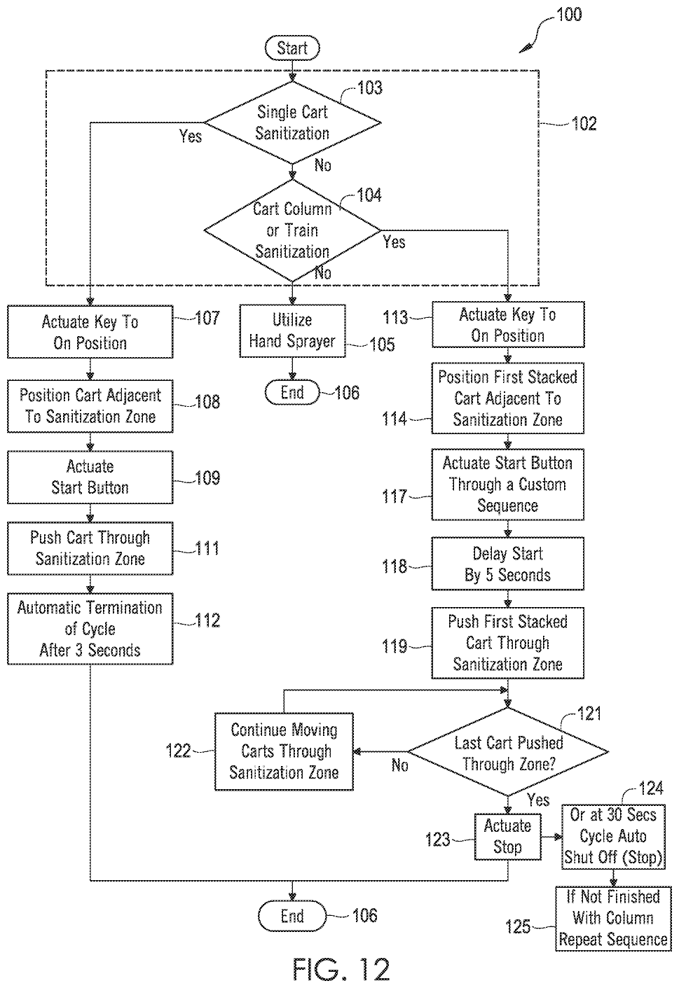

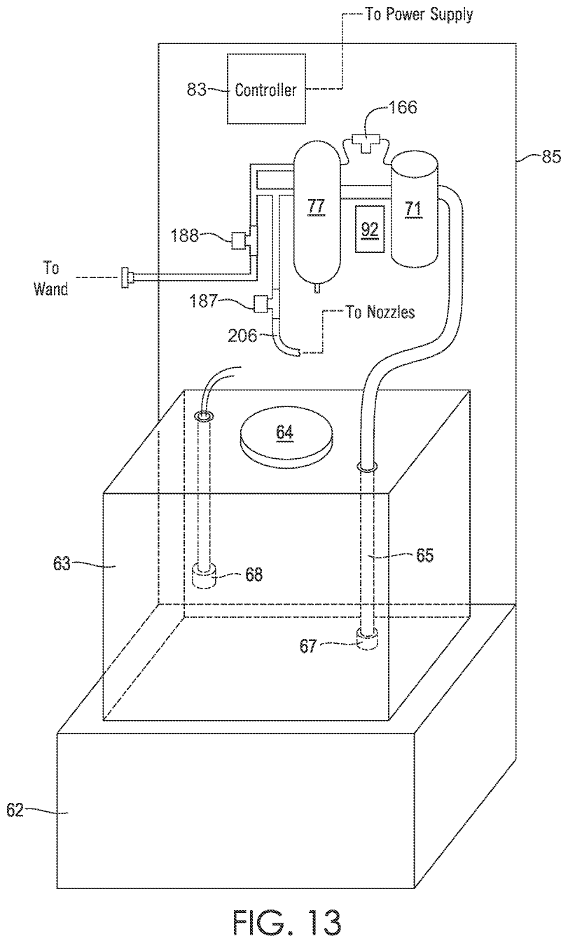

In other embodiments, such as is shown in FIGS. 10A, 10B and 13, a central solenoid 187 may control flow to the left, right and upper nozzles 86, and another solenoid 188 may control flow to the hand sprayer 56. The pump 71, accumulator 77, controller 83, hand sprayer solenoid 188, nozzles solenoid 187, and a pump relay 79 are all mounted on a removable base 85 that projects up from the frame 62 to provide for a centralized location for the pumping system. In some embodiments, the removable base 85 is a thin, flat structure, referred to herein as a “backpan.” In other embodiments, other configurations of the removable base 85 are possible.

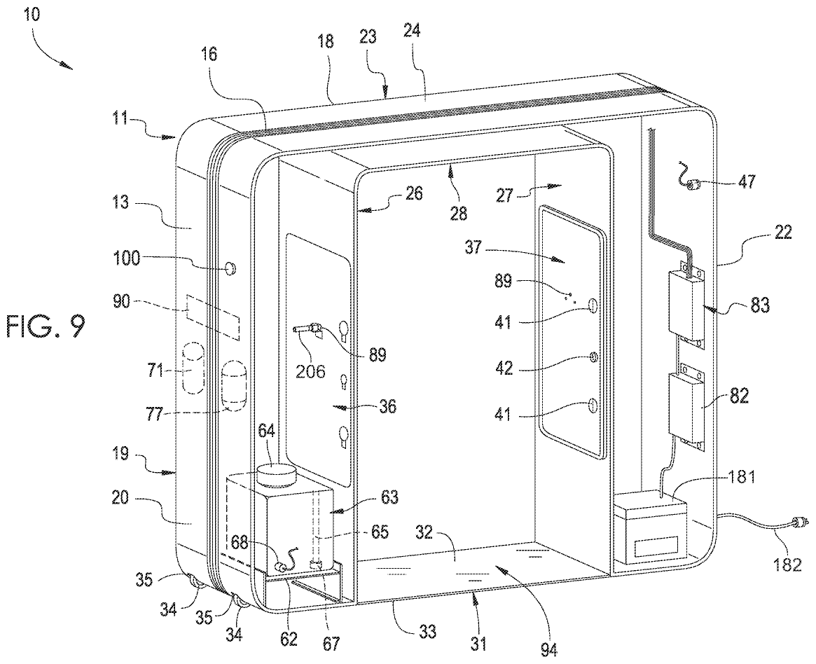

As shown by FIG. 13, an inline strainer 166 is coupled between the pump 71 and the accumulator 77 to prevent particulate matter from clogging the solenoids 187, 188 and/or nozzles 86. In this regard, fluid from the pump 71 passes through the strainer 166 to the accumulator 77, and the strainer 166 has an internal mesh screen that filters particles from the fluid flowing through the strainer 166. Referring to FIG. 9, a power supply 181 feeds power to the controller 83 which then feeds power as may be necessary or desired to the pump 71, accumulator 77 and solenoids 187, 188. The power supply 181 may be fed power by an alternating current (AC) signal by a cable 182 plugged into an external electrical outlet (not shown), or the power supply 181 may comprise or be coupled to a battery that a direct current (DC) power signal. In other embodiments, other types of power supplies may be used. As an example, it is possible for the system 10 to include one or more solar power panels for converting sunlight to electrical power that is processed by the power supply 181.

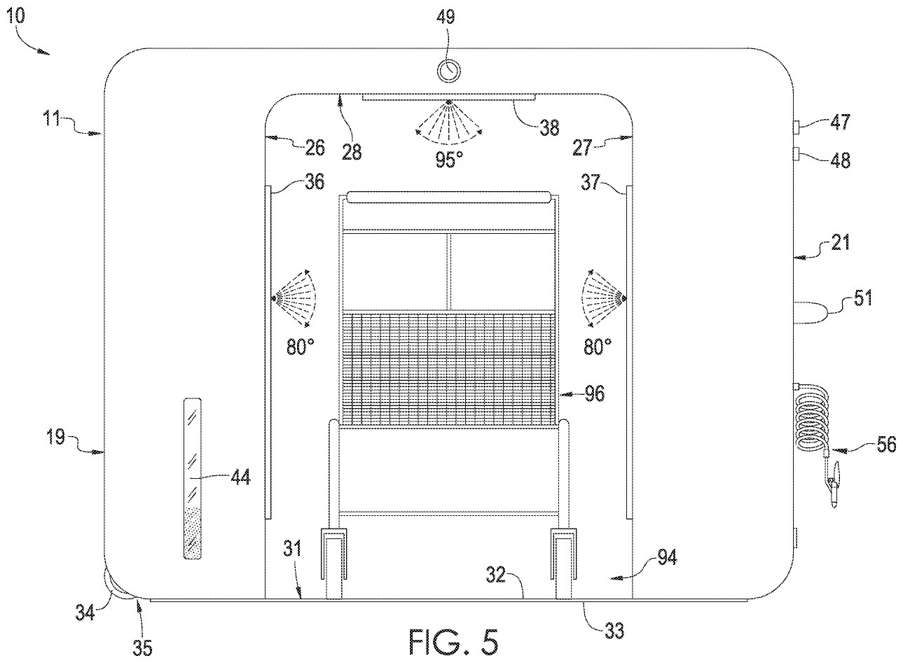

Exemplary dynamics of sanitizing a cart 96 may be seen in FIG. 5 with reference to the system 10 shown by FIG. 1. As discussed previously, nozzles 86 are positioned around the sanitization zone 94 defined by the housing 11 to create a disinfection zone when solenoids actuate nozzles 86. The accumulator 77 maintains a pressure of between about 65 and 100 psi inside all tubing that connects each spray nozzle 86 to accumulator 77. The spray nozzle 86 positioned in upper access door 38 is configured to provide a fan type spray pattern with a dispersion angle of between about 95-110 degrees to accommodate a number of different items that can be disinfected including shopping carts, wheel chairs, suit cases, tool boxes that shift workers use between shifts and a variety of other items that can carry and transmit disease to a future user. At between about 65 and 100 psi, nozzle 86 in door 38 will provide an approximate flow volume of between about 0.040 and 0.052 gallons per minute, at a maximum particle size of approximately 175 microns.

The nozzles 86 on the left and right access doors 36, 37 are also configured to provide a fan type spray pattern with a dispersion angle of about 80 degrees. In other embodiments, the nozzles 86 on the left and right access doors 36, 37 may be configured to spray at other dispersion angles, such as between about 80-110 degrees. At between about 65 and 100 psi, the nozzles 86 in left and right access doors 36, 37 provide an approximate flow volume of between about 0.031 and 0.040 gallons per minute, at a maximum particle size of approximately 150 microns. In some embodiments, the shopping cart may be passed through the sanitization zone 94 at about one foot per second or some other rate as may be desired. The configuration and placement of the spray nozzles 86 around the sanitization zone 94, at the above specified flow and particle sizes, and speeds, helps to maximize surface coverage of a shopping cart or other item being passed through the zone 94. Note that the pressures, flow rates, dispersion angles, and other operational parameters described above are exemplary, and other values for these parameters are possible.

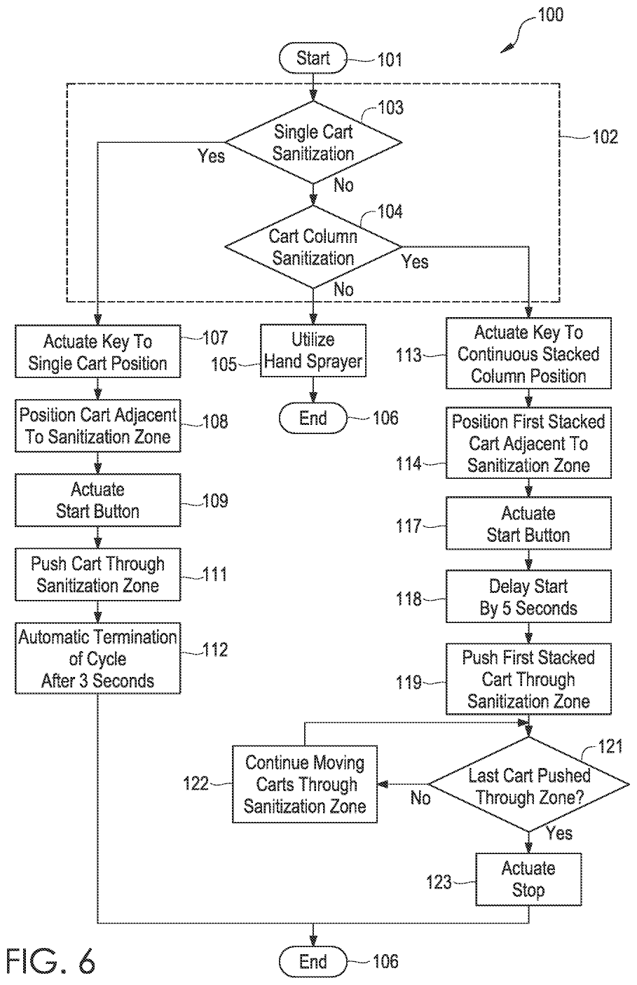

Referring now to FIG. 6, it may be seen that, in at least some embodiments, three principle modes may be utilized in system 10 in sanitizing a shopping cart or basket, as shown in block 102. After initiating the system 10, the system 10 may either be used to sanitize a single cart, as shown by block 103, or an entire collapsed or folded cart column, as shown by block 104. A cart column is a series of shopping carts grouped and engaged into an interlocking and stacked configuration to save space, as is known by any grocery worker. If neither of these cart configurations is to be sanitized by the system 10, then the hand sprayer 56 may be to be used 105 to sanitize either a single cart or other carrying container or item, such as a hand basket.

If a single cart is to be sanitized, three position key switch 47 is turned to the appropriate mode in block 107 and the cart is positioned adjacent to the sanitization zone 94, as shown by block 108. As shown by block 109, the actuation switch 49 is controlled to provide an input for activating the solenoids 87 to initiate spray from nozzles 86 into zone 84. For illustrative purposes, it will be assumed hereafter that the switch 49 comprises a button that may be pressed by a user to provide an input, such as when to start or stop spraying, and this switch 49 will be referred to herein as a “start-stop button.” However, it should be emphasized that other types of switches may be used in other embodiments, and the description of the switch 49 as a start-stop button is exemplary.

Once the start-stop button is pressed to indicate that spraying is to commence, the cart is pushed through the sanitization zone 94 at approximately 1 foot per second speed, as shown by block 111. As the cart is passes through the zone, all surface areas will be covered and sanitized with disinfecting fluid from the tank 63 sprayed by the nozzles 86. The controller 83 keeps track of the expended flow time during fluid release and shuts off each spray nozzle 86 via solenoids 87 after three seconds have elapsed, as shown by block 112. After the cart has passed through the zone 94, the cycle ends 106 in response to the expiration of the three seconds. In other embodiments, the spray nozzles 86 may be activated for other durations of time or the timing of the activation of the spray nozzles 86 may be controlled in other ways. As an example, the system 10 may have one or more proximity sensors (not shown) or other types of sensors for sensing when the item or items being sanitized have completely passed through the zone 94 or reached a predefined location, and spray from the nozzles 86 may be deactivated when such a point is reached.

In an alternate embodiment, the key switch 47 has an on position, an off position and a maintenance position. When the key switch 47 is in the on position, a user may activate the system 10 for treatment of an individual cart by quickly pressing and releasing the start-stop button 49 once. To activate the system 10 to treat a cart column or cart train, the user may press the start-stop button 49 for at least a predefined period of time or press the start-top button 49 for at least a certain number of times within a predefined period of time. One example would be for a user to press the start-stop button 49 for a period of at least five seconds before releasing the start-stop button 49. In response to such an input, the controller 83 may be configured to then actuate the system 10 to spray the shopping cart column for a predefined period of time, such as about 30 seconds, before shutting off the spraying. A user can actuate the system 10 again by pressing the start stop button 49 again for at least five seconds (or some other predefined period of time) and can at any time after actuation press the start-stop button 49 to stop the system 10 from spraying sanitizer.

To activate the hand sprayer 56, a user may press and hold both the start-stop button 49 and a hand sprayer button 100 located on the side of the housing 11 for at least a preset period of time, such as about 5 seconds for example. In response, the controller 83 actuates the hand sprayer solenoid 87 such that a user can use the hand sprayer 56 to disinfect surfaces that are not suited to be run through the housing 11. When the key switch 47 is turned to the maintenance setting, the hand sprayer 56 is activated such that it can be used to prime the accumulator 77 when the system 10 is being used for the first time by running the hand sprayer 56 for a set period of time. In a further embodiment, there is a start-stop button 49 on each side of the system housing 11 such that a user can activate the system 10 whether they are on the front or rear of the system 10. In a further embodiment, a digital touch screen can replace the key switch 47, start stop-button 49 and hand sprayer button 100 such that user can have a centralized location of control with various options and benefits that are inherent to digital touch screen interfaces. Yet other configurations and techniques for providing input the system 10 for activating and deactivating spraying are possible. As an example, it is possible for a device, such as a smartphone or other handheld device to communicate with the controller 83 remotely, and an interface of the smartphone or handheld device may be used to receive user inputs that are wirelessly communicated to the controller 83.

A cart column can also be sanitized with equally successful results. If a column is to be sanitized 104, the key switch 47 may be turned to a continuous or column mode, as shown by block 113. The stacked carts are then moved such that the first cart is adjacent to the sanitization zone 94, as shown by block 114. The start-stop button 49 is depressed, as shown by block 117, and the solenoids are opened after a 5 second delay (or some other period), as shown by block 118. The nozzles 86 then begin to spray fluid. The first stacked cart is then pushed through the sanitization zone 94 and the entire cart column is moved at approximately 1 foot per second through the zone 94 until all of the carts have been moved through the system 10, as shown by blocks 119, 121-122. After the last cart has been pushed through, the key switch 47 is rotated to its off position to turn off system 10 or the start-stop button is again depressed to stop the cycle, as shown by block 123. The start-stop button 49 may be repeatedly depressed to toggle on and off the spray nozzles as needed to position and re-position stacked carts through the sanitization zone 94. This ends the stacked column sanitization process. Additionally, controller 83 can be programmed or otherwise configured such that the column mode shuts off automatically after thirty seconds or other pre-determined time after activation of spraying, as shown by block 124. Optionally, a user can repeat the column mode as may be desired, as shown by block 125. As will be described in more detail below, once a particular spraying mode ends, such as a single-cart mode or column mode, the controller 83 transitions to a mode, referred to herein as “depressurization mode,” in which the tubing 206 connected to the nozzles 86 is depressurized (e.g., lowered to around atmospheric pressure well below the pressure maintained by the pump 71).

In either the single cart or column mode, spraying may be stopped in response to a certain user input, such as pressing of the button 49. That is, while the system 10 is spraying, the controller is configured to stop spraying and end the current mode of operation in response to activation of the button 49 during spraying. After controller 83 stops the system 10 from spraying in response to such an input, the controller 83 may wait for further input, such as an input to commence the single cart or column mode, as described further herein.

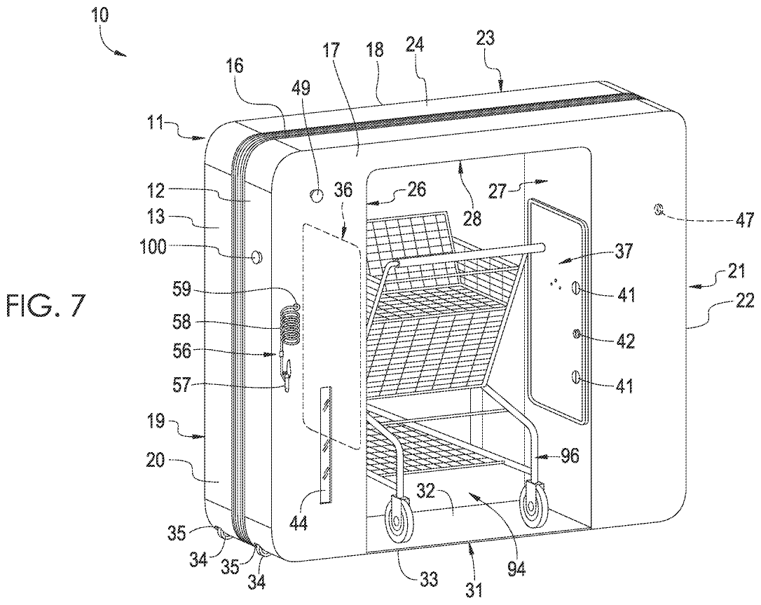

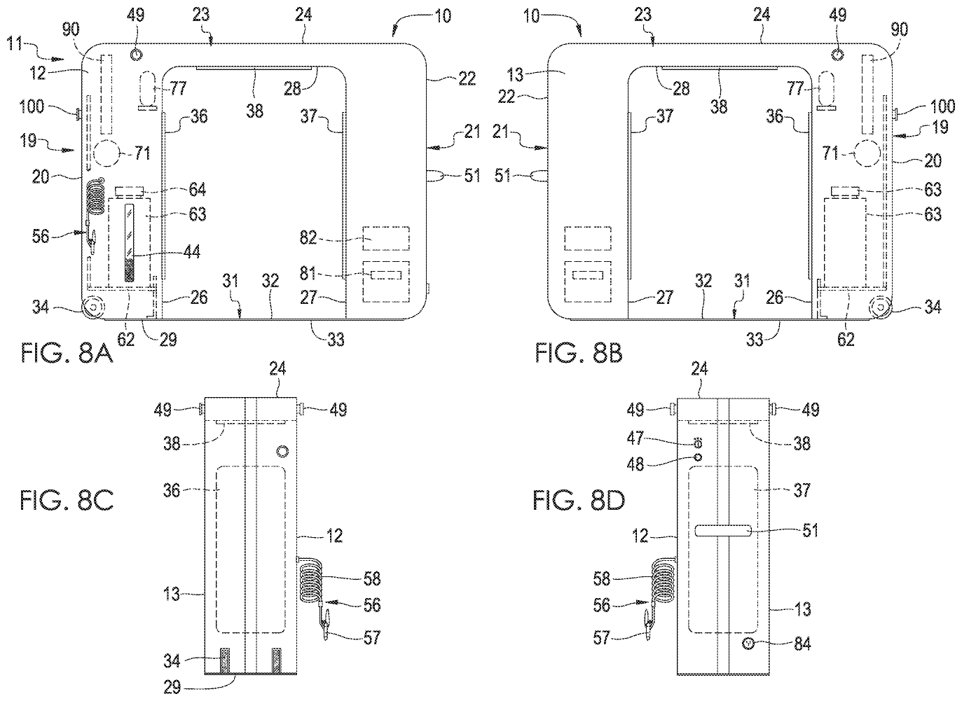

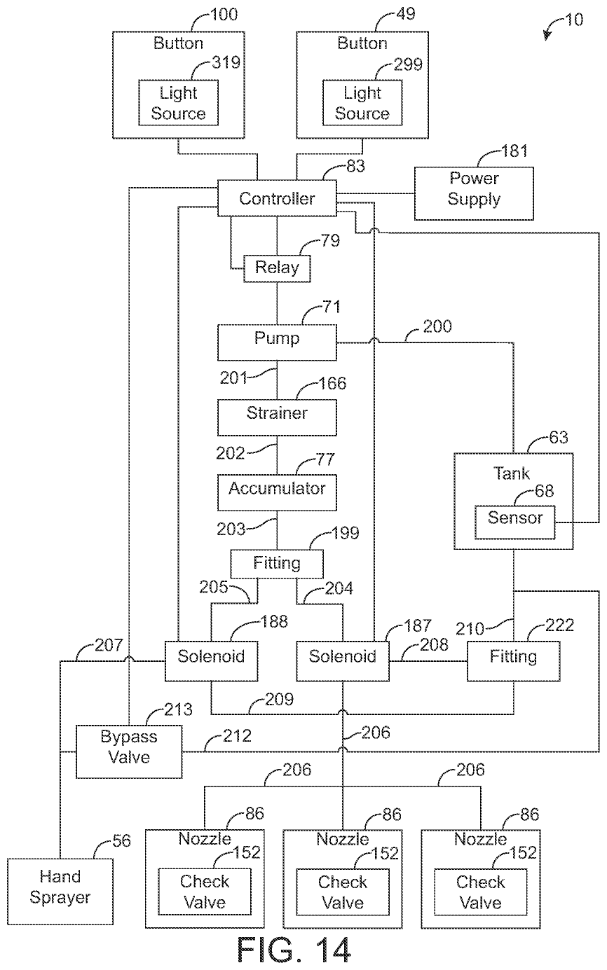

FIG. 14 is a block diagram of various components for an exemplary embodiment of a sanitization system 10. For illustrative purposes, it will be assumed hereafter unless otherwise indicated that the components of FIG. 14 are situated within the housing 11 shown by FIGS. 7, 8A, 8B, 8C, 8D, and 11.

As shown by FIG. 14, the controller 83 is electrically coupled to and receives electrical power from the power supply 181, and the controller 83 is also electrically coupled to the pump 71 through a relay 79, referred to hereafter as “pump relay.” The controller 83 is configured to transmit a power signal through the pump relay 79 for powering the pump 71. The controller 83 is also configured to transmit a control signal to the pump relay 79 for controlling the state of the relay 79. As an example, when the pump 71 is to be activated, the controller 83 is configured to close the relay 79 such that the power signal from the controller 83 passes through the relay 79 and is received by the pump 71. In such state, the pump 71 is turned on and attempts to maintain the pressure in the accumulator 77 within a predefined range. When the pump 71 is to be deactivated, such as in response to a fault condition or low fluid level in the tank 63, the controller 83 is configured to transition the pump relay 79 to an open state such that the power signal from the controller 83 is prevented from passing through relay 79. In such a state, the pump 71 is turned off such that it no longer maintains the pressure in the accumulator 77 until the relay 79 is later closed by the controller 83. As shown by FIG. 14, the controller 83 is also electrically coupled to the float sensor 68 thereby enabling the controller 83 to determine the level of fluid in the tank 63 based on readings from the float sensor 68.

Further, the pump 71 is coupled to the tank 63 by hollow tubing 200 and the inline strainer 166 by hollow tubing 201, and the strainer 166 is coupled to the accumulator 77 by hollow tubing 202. The accumulator 77 is coupled to an inlet of fitting 199 by hollow tubing 203, and outlets of the fitting 199 are respectively coupled to the solenoids 187, 188 by hollow tubing 204, 205. The fitting 199 is designed to separate the flow of fluid from the accumulator 77 into separate flows to the solenoids 187, 188. The solenoid 187 is coupled to each nozzle 86 through hollow tubing 206, and the solenoid 188 is coupled to the hand sprayer 56 and a bypass valve 213 by hollow tubing 207. In addition, each of solenoids 187, 188 is coupled to a fitting 222 by hollow tubing 208, 209, respectively, and the fitting 222 is coupled to the tank 63 by hollow tubing 210. The fitting 222 is designed to combine flows from the solenoids 187, 188 into a single flow to the tank 63. The bypass valve 213 is coupled to the tank 63 by hollow tubing 212. The bypass valve 213 operates in either an open state in which fluid may flow through the bypass valve 213 to the tank 63 or a closed state in which fluid is prevented from flowing through the bypass valve 213.

As shown by FIG. 14, the controller 83 is electrically coupled to each of the solenoids 187, 188 and the bypass valve 213 for controlling these components, as will be described in more detail below. In some embodiments, the bypass valve 213 may be manually controlled instead of being controlled by the controller 83. As an example, the bypass valve 213 may be configured to be actuated by hand to transition between the open state and the closed state as may be desired. During the maintenance mode, the bypass valve 213 may be used to help depressurize the accumulator 77 and empty fluid from the accumulator 77 into the tank 63. In this regard, the bypass valve 213 normally operates in the closed state to allow fluid to flow through tubing 207 under pressure to the hand sprayer 56. In the maintenance mode, the controller 83 may control the bypass valve 213 or the bypass valve 213 may be manually controlled to transition it to the open state such that fluid instead bypasses the hand sprayer 56 and flows through the bypass valve 213 to the tubing 212, which leads to the tank 63. Thus, fluid from the accumulator 77 empties to the tank 63 through the fitting 199, solenoid 188, and bypass valve 213.

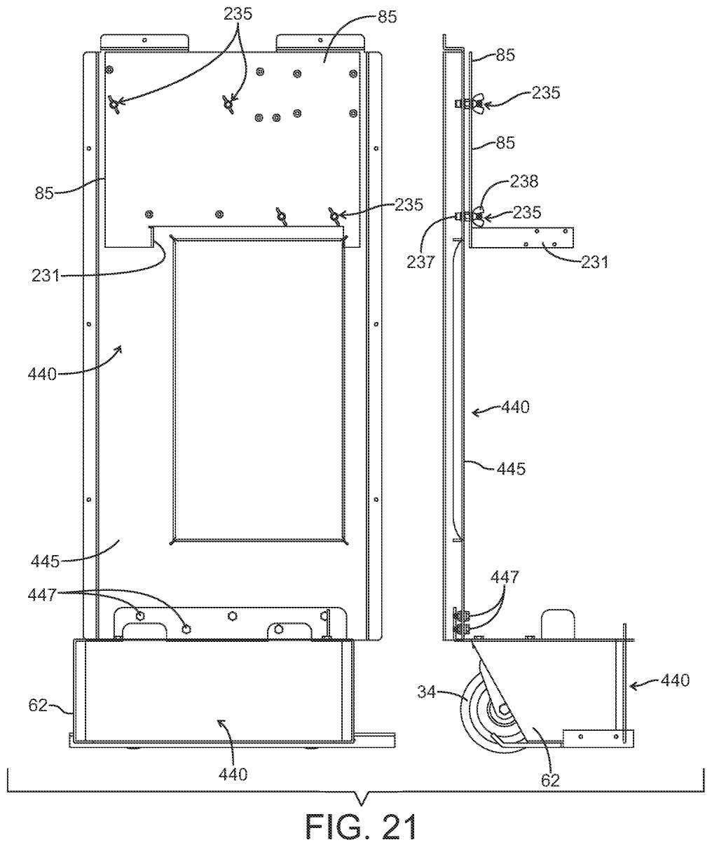

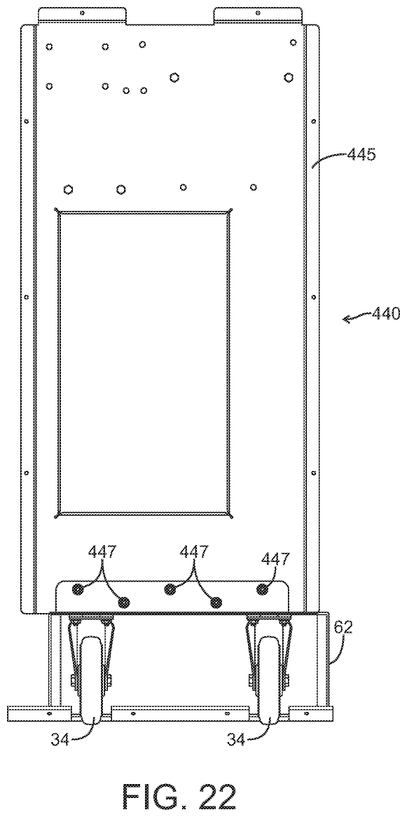

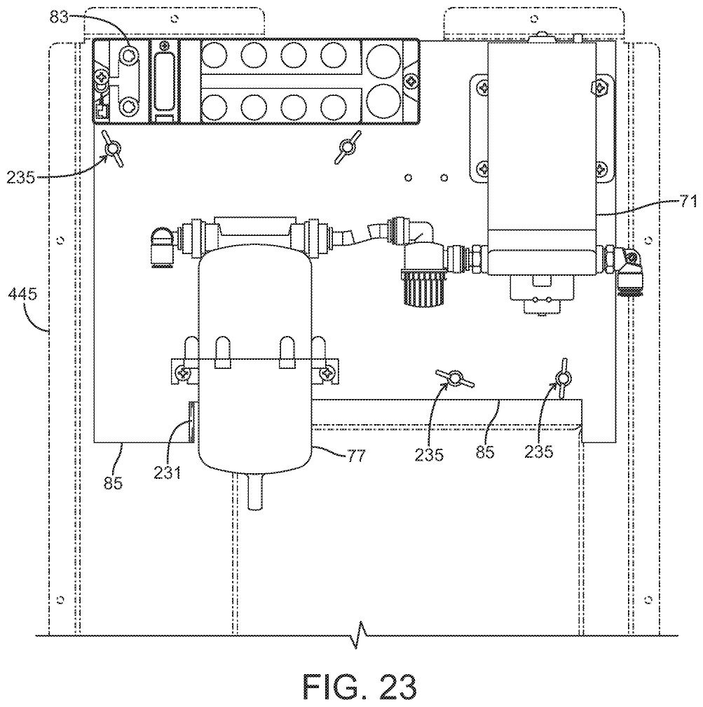

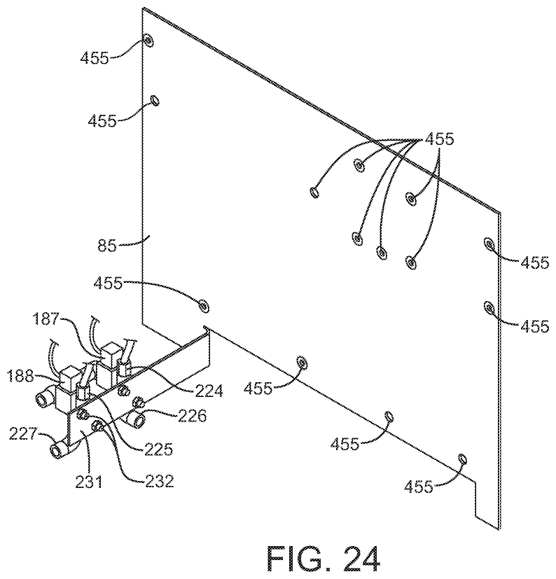

As shown by FIGS. 23 and 24, the accumulator 77 and solenoids 187, 188 are mounted on a removable base 85, as described above. In this regard, a bracket 231 may be coupled to and/or extend from the removable base 85, and each solenoid 187, 188 is mounted on the bracket 231 by one or more couplers 232, such as screws or bolts. Further, the removable base 85 may be mounted on an inner wall of the housing 11 or a structure within the housing 11 by a plurality couplers 235, such as screws or bolts. FIGS. 21 and 22, which will be described in more detail below, show an exemplary structure on which the removable base 85 may be mounted. In the embodiment shown by FIG. 21, each coupler 235 comprises a threaded bolt 237 that extends from the inner wall of the housing 11 or other structure within the housing 11. A nut 238, such as a winged nut, is screwed on the threaded bolt 237. When the base 85 is to be removed from the system 10, the pump 71 may be detached from the tubing 200, and the solenoids 187, 188 may be detached from the tubing 206-209. Further, the nuts 238 may be removed (e.g., unscrewed) from the bolts 237, and the base 85 may be pulled off the bolts 237 and removed from the housing 11, as will be described in more detail below.

As shown by FIG. 14, each nozzle 86 comprises a check valve 152 that prevents fluid flow of a pressure below a certain threshold. That is, when the pressure in the tubing 206 is less than a certain threshold, the check valve 152 is closed thereby blocking fluid from flowing through the check valve 152 and thus preventing the nozzle 86 from emitting fluid. However, when the pressure in the tubing 206 exceeds the threshold, the check valve 152 is opened thereby allowing fluid to flow through the check valve 152 and be emitted from the nozzle 86. In some embodiments, the check valve 152 has a spring-loaded plunger (not shown) that moves into position to block fluid flow through the check valve 152 when pressure is below the threshold. When the threshold is exceeded, the pressure in the tubing 206 is sufficient to overcome the force of the spring (not shown) holding the plunger in position, such that the plunger is moved to allow fluid to flow past the plunger. Check valves are generally well-known devices, and any conventional check valve may be used in the nozzles 86 as may be desired. In some embodiments, the check valve 152 is designed to transition to an open state at a pressure of about 40 psi, but other thresholds for the check valve 152 are possible in other embodiments. In addition, in other embodiments, the check valves 152 may be at locations other than in the nozzles 86. As an example, an inline check valve may be used between a nozzle 86 and the solenoid 187 to control fluid flow to the nozzle 86. Locating a check valve 152 at or near nozzle 86 helps to reduce dripping, as will be described in more detail below.

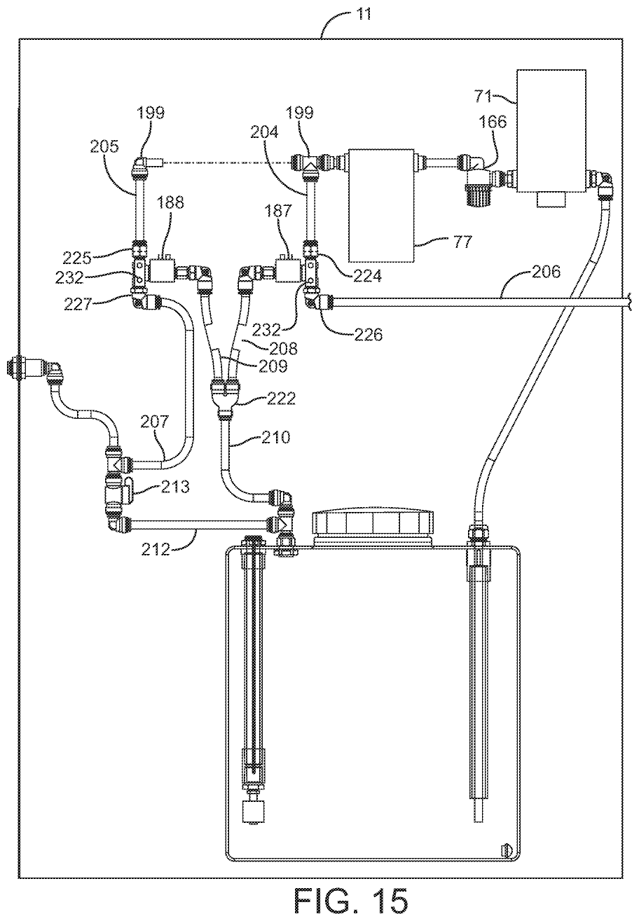

During operation, the pump 71 is configured to pump fluid out of the tank 63 and through the inline strainer 166 to the accumulator 77 thereby filling the accumulator 77 with fluid, and fluid from the accumulator 77 flows to the solenoids 187, 188. Note that in FIG. 15, the fitting 199 is shown as separate structures so that the spacing of the solenoids 187, 188 can be shown to be spread out for illustrative purposes. However, the solenoids 187, 188 may be positioned next to each other and mounted on a bracket as shown by FIG. 24, as will be described in more detail below. Further, each solenoid 187, 188 may implement a three-way valve that allows fluid to selectively flow through three ports, as described in more detail below, though other types of solenoids may be used in other embodiments.

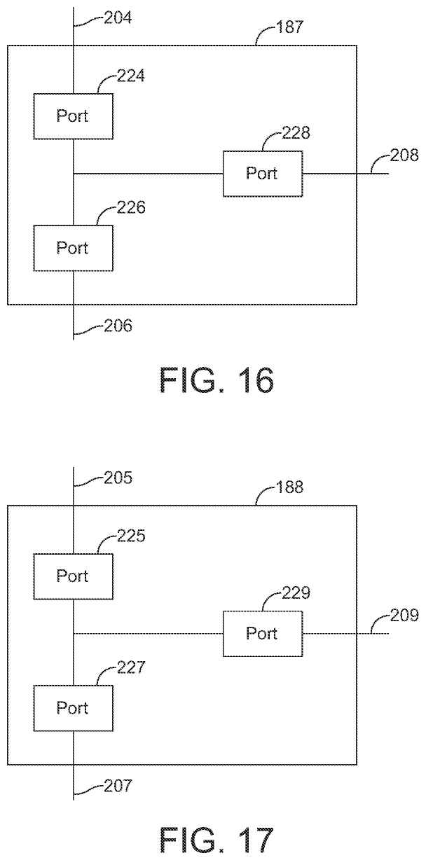

Specifically, as shown by FIG. 16, the solenoid 187 has a port 224 coupled to the tubing 204 from the fitting 199, a port 226 coupled to the tubing 206 to the nozzles 86, and a port 228 coupled to the tubing 208 to the fitting 222. In addition, the solenoid 187 is electrically coupled to the controller 83 and operates under the direction and control of the controller 83 to selectively open and close the ports 224, 226, 228. Specifically, the port 226 may be controlled to remain open at all times so that fluid may flow through it. When the nozzles 86 are to be activated for spraying, the controller 83 controls the solenoid 187 such that the port 224 is opened, thereby permitting fluid to flow through the port 224, and the port 228 (referred to hereafter as “bypass port”) is closed, thereby preventing fluid from flowing through the bypass port 208. Thus, the accumulator 77 with pressurization by the pump 71 builds pressure within the tubing 206 running to the nozzles 86 forcing sanitizing fluid to flow from the accumulator 77 to the nozzles 86. Such pressure is sufficiently strong to open the check valves 152 at the nozzles 86, and the nozzles 86 begin to spray the sanitizing fluid.

When spraying by the nozzles 86 is to be deactivated (e.g., when the controller 83 transitions to the depressurization mode at expiration of the single-cart mode or the column mode), the controller 83 controls the solenoid 187 such that the port 224 is closed, thereby preventing fluid from flowing through the port 224, and the bypass port 228 is opened, thereby permitting fluid to flow through the bypass port 228. In this state, referred to hereafter as the “bypass state,” the pressure from the pump 71, that is stored in the accumulator 77, is cutoff by the closed port 224 of the solenoid 187, and the pressure in the tubing 206 running to the nozzles 86 thus is permitted to bleed down. Specifically, the relatively high pressure in the tubing 206 induced by the pump 71 and accumulator 77 is released through the bypass port 228 to the tank 63, thereby lowering the pressure in the tubing 206. Further, fluid in the tubing 206 is pulled by the pressure change and gravity into the tank 63. Thus, the nozzles 86 are depressurized, and the excess fluid in the tubing 206 substantially empties into the tank 63 and may be re-used later when spraying is activated in the future. In addition, due to the depressurization of the tubing 206, the check valves 152 at the nozzles 152 transition to a closed state thereby preventing fluid from flowing through the check valves 152 and thus preventing the nozzles from leaking.

Note that the use of a single solenoid 187 with three ports in order to depressurize the tubing 206 is unnecessary. As an example, it possible to use multiple solenoids for which one solenoid (not shown) is connected between the accumulator 77 and the tubing 206 for controlling whether the pump 71 pressurizes the tubing 206 and another solenoid (not shown) is connected between the tubing 206 and the tank 63 for controlling when the fluid is permitted to bypass the accumulator 77 and flow into the tank 63 to depressurize the tubing 206. Other configurations for allowing the tubing 206 to be depressurized are possible in other embodiments.

In addition, it should also be noted that, in some embodiments, more than one tank 63 may be used. As an example, one tank 63 may be connected to the pump 71 so that fluid may be pulled out of such tank 63 by the pump 71, and another tank (not shown) may be connected to the bypass port 228 to collect fluid that flows from the tubing 206 when it is depressurized. Yet other numbers of tanks and configurations are possible in other embodiments.

The aforementioned depressurization of the tubing 206 has many potential advantages relative to an embodiment, such as the system 10 depicted by FIG. 1, where an individual solenoid is used at or near each nozzle 86. In addition, at least some of these benefits are enhanced when the depressurization is achieved through control of a central solenoid 187 located away from the nozzles 86 and close to the accumulator 77 (e.g., closer to the accumulator 77 than the nozzles 86). As an example, if there is a leak at any nozzle 86 or in the tubing 206 or connection points for the tubing 206, then the depressurization should eliminate or at least reduce the amount of fluid that leaks while spraying remains deactivated. Thus, the likelihood of a large leak or spill, such as leaks or spills that might accumulate or occur overnight without being noticed, is significantly reduced.

In addition, the depressurization of the nozzles 86 during deactivation of spraying (e.g., during the depressurization mode) facilitates maintenance of the system 10. In this regard, while spraying is deactivated, a user may clean the nozzles 86 and/or tips of the nozzles 86 without having to access the inner region of the housing 11 in order to turn off the pump 71 or interface with the controller 83. As an example, a nozzle 86 or nozzle tip may be unscrewed or otherwise removed, cleaned, and then replaced. Since the tubing 206 is depressurized, the maintenance on or cleaning of the nozzles 86 will not cause fluid to be expelled from the system 10 at the nozzle locations. In addition, once the depressurization sufficiently reduces the pressure in the tubing 206 to actuate the check valves 152, the check valves 152 stop fluid flow, thereby preventing dripping of fluid from the nozzles 86 that would otherwise occur as the pressure continues to bleed down. Thus, once spraying is deactivated, there should be less dripping from the nozzles 86 relative to an embodiment that does not use a check valve at or close to the nozzles 86.

Note that the configuration and use of the solenoid 188 may be similar to the configuration and use of the solenoid 187. Specifically, as shown by FIG. 17, the solenoid 188 has a port 225 coupled to the tubing 205 from the fitting 199, a port 227 coupled to the tubing 207 to the hand sprayer 56 and bypass valve 213, and a port 229 coupled to the tubing 209 to the fitting 222. In addition, the solenoid 188 is electrically coupled to the controller 83 and operates under the direction and control of the controller 83 to selectively open and close the ports 225, 227, 229. Specifically, the port 227 is controlled to remain open at all times so that fluid may flow through the port 227. When the hand sprayer 56 is to be activated for spraying, the controller 83 controls the solenoid 188 such that the port 225 is opened, thereby permitting fluid to flow through the port 225, and the port 229 (referred to hereafter as “bypass port”) is closed, thereby preventing fluid from flowing through the bypass port 229. Thus, the pump 71 builds pressure within the tubing 207 running to the hand sprayer 56 and bypass valve 213 forcing sanitizing fluid to flow from the accumulator 77 to the hand sprayer 56 when spraying by the hand sprayer 56 is activated. Thus, when the handle 57 of the hand sprayer 56 is actuated by a user, the pressure within the tubing 207 causes sanitizing fluid to be sprayed by the sprayer 56.

When spraying by the sprayer 56 is to be deactivated, the controller 83 controls the solenoid 188 such that the port 225 is closed, thereby preventing fluid from flowing through the port 225, and the bypass port 229 is opened, thereby permitting fluid to flow through the bypass port 229. In this bypass state, the pressure from the pump 71 is cutoff by the closed port 225 of the solenoid 188, and the pressure in the tubing 207 running to the bypass valve 213 and the hand sprayer 56 thus is permitted to bleed down. Specifically, the relatively high pressure in the tubing 207 is released through the bypass port 229 to the tank 63, thereby lowering the pressure in the tubing 207. Further, fluid in the tubing 207 is pulled by the pressure change and gravity into the tank 63. Thus, the tubing 207 is depressurized, and the excess fluid in the tubing 207 substantially empties into the tank 63 and may be re-used later when spraying is activated in the future.

In some embodiments, the controller 83 is configured to monitor the operation of the system 10 to detect possible leaks or other faults and to take at least one action to mitigate the detected leak or fault, such as deactivating the pump 71. As an example, when a leak is occurring in the system 10, such as in the strainer 166, the accumulator 77 or the tubing connecting the strainer 166 and accumulator 77 to other components of the system 10, such leak may cause the pump to 71 turn on frequently. In this regard, when receiving power from the controller 83 through the relay 79, the pump 71 is configured to sense pressure at the output of the pump 71 and may be configured to maintain the sensed pressure above a predefined lower threshold. If the pressure sensed by the pump 71 falls below the lower threshold, the pump 71 may turn on to increase pressure until the pressure sensed by the pump 71 reaches an upper threshold at which point the pump 71 then turns off.

In the absence of leaks, it is generally expected that the sensed pressure should be substantially maintained above the lower threshold until at least spraying is activated. However, a leak in the system 10 may cause the pressure to slowly bleed down such that the sensed pressure may fall below the lower threshold causing the pump 71 to turn on. Thus, activation of the pump 71 for short durations while spraying is deactivated may indicate that a leak exists between the pump 71 and the solenoids 187, 188. Thus, in some embodiments, the controller 83 is configured to track the operation of the pump 71 to determine whether the activation profile (e.g., frequent activation of the pump 71 over a defined time period) of the pump 71 is indicative of a likely leak or other fault.

Note that there are various techniques that may be used to determine whether the activation profile of the pump 71 or other components of the system 10 is indicative of a likely leak or other operational fault. In some embodiments, the controller 83 is configured to count the number of times that the pump 71 is activated over a defined time period, such as ten minutes for example, while spraying is deactivated and to compare the count to a predefined threshold (e.g., three or some other number). If the threshold is exceeded, then the controller 83 is configured to detect a likely leak or other operational fault and take at least one action to notify a user of the fault or mitigate the fault. As an example, the controller 83 may be configured to detect a likely leak if the pump 71 is activated three (or some other number) or more times within any 10 minute window while spraying is deactivated. In other embodiments, other thresholds and time periods may be used.

If a likely leak or other operational fault is detected, the controller 83 may be configured to deactivate the pump 71 and provide a notification of the detected operational fault. As an example, if the controller 83 is communicatively coupled to a communication network (e.g., a cellular network and/or the Internet), the controller 83 may send a message through the network to a user in order to notify him or her of the detected fault. In some embodiments, the system 10 may have a user interface, such as a display screen, a speaker, or a light source, that is controlled by the controller 83 to notify a user in a vicinity of the system 10 of the detected fault. In some embodiments, the controller 83 is configured to keep the pump 71 in a deactivated state until a user has inspected the system 10 and provided an input indicating the normal operation is to resume. By deactivating the pump 71 in response to a detected fault, such as a leak, the controller 83 may prevent at least some fluid from leaking from the system 10 until the system 10 has been inspected. In other embodiments, other actions may be taken by the controller 83 in response to a detected fault.

Note that there are various techniques that may be used by the controller 83 to determine when the pump 71 is activated for the purpose of monitoring the operational state of the pump 71. As an example, it is possible for the pump 71 to be configured to communicate with the controller 83 for the purpose of informing the controller 83 of its operational state. In some embodiments, such as the embodiment shown by FIG. 14, electrical power may be supplied to the pump 71 from the power supply 181 through the controller 83. In such an embodiment, the controller 83 may monitor the power signal supplied to the pump 71 by the controller 83 to determine the operational state of the pump 71. In this regard, when the pump 71 activates, the amount of power drawn by the pump 71 significantly increases, and such power significantly decreases when the pump 71 is deactivated. The controller 83 may be configured to detect an activation of the pump 71 when the power signal provided to the pump 71 increases above a predefined threshold, and the controller 83 may be configured to detect a deactivation of the pump 71 when the power signal provided o the pump 71 falls below a predefined threshold. In other embodiments, yet other techniques for monitoring the operational state of the pump 71 are possible.

In addition, other techniques may be used to detect a likely leak or other operational fault based on the operation of the pump 71. For example, the controller 83 may be configured to detect an operational fault, such as a leak, if the pump 71 continuously runs for at least an amount of time above a predefined threshold while spraying is deactivated. In this regard, if spraying is not occurring, it is generally expected that the pump 71 should remain deactivated. However, over time, some decrease in pressure in the tubing between the pump 71 and the solenoids 187, 188 is expected due to imperfect seals. Thus, it is possible in normal operation for the pressure to fall below the lower threshold monitored by the pump 71 such that the pump 71 briefly activates to raise the pressure back above the lower threshold. In the absence of a leak, it is expected that the amount of time to raise the pressure to account for normal depressurization should be short. Thus, if the pump 71 runs for a relatively long time, such as more than just a few seconds while spraying is deactivated, then it is possible that an unexpected leak is causing a greater amount of depressurization than what would be considered normal. In such a situation, the controller 83 may detect an occurrence of leak.

As an example, if the controller 83 determines that the pump 71 is activated and remains continuously activated for at least a predefined amount of time (such as about 10 seconds for example) while spraying s deactivated (e.g., while operating in the depressurization mode), then the controller 83 may be configured to detect an occurrence of an operational fault, such as leak, and take corrective action as described above (e.g., notify a user of the detected fault and/or deactivate the pump 71 until it is inspected and an input is received indicating that normal operation is to resume). In other embodiments, other techniques for detecting operational faults based on the activation profile of the pump 71 are possible.

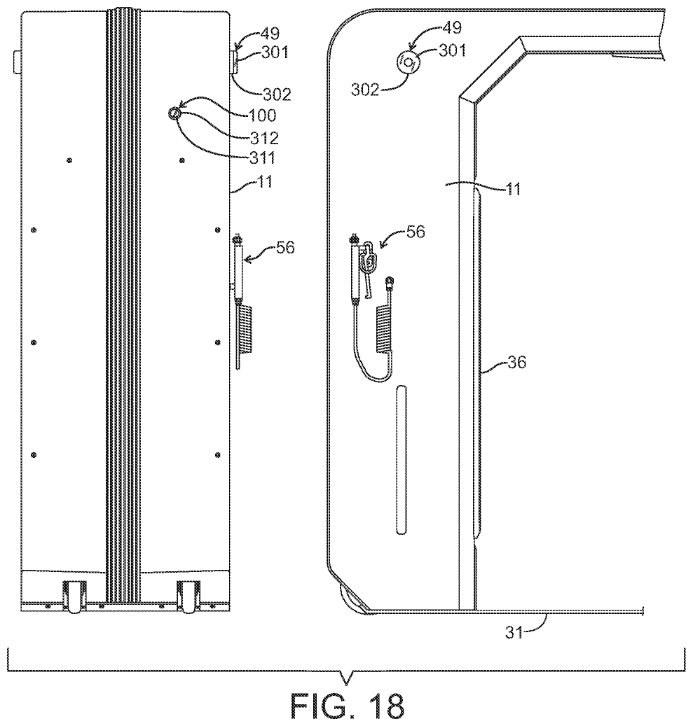

It should be noted that there are various types of user interfaces that may be used to convey information from the system 10 to a user. In some embodiments, information about the operation of the system 10, such as the type of mode in which the system 10 is currently operating, is conveyed by at least one button 49. In this regard, the start-stop button 49 may be arranged to have at least one light source 299, such as one or more light emitting diodes (LEDs), as shown by FIGS. 14 and 18. In this regard, the button 49 comprises a frame 302 on which a translucent cover 301 is positioned. Such cover 301 may be spring loaded such that it moves in response to a press by a user for sensing such press and returns to its original position when the user stops pressing on the cover 301. The light source 299 is positioned within a cavity formed by the frame 302 and the cover 301 such that light from the light source 299 passes through the cover 301 and is visible to a user. Thus, the cover 301 appears to be illuminated at the color of the light from the light source 299. In some embodiments, the button 49 has multiple LEDS or other types of light sources 299 that emit different colors (e.g., red, green and blue) so that the color of the cover 301 may be changed as may be desired. As an example, as will be described in more detail below, the cover 301 may be color coded to indicate certain operational information to a user.

As an example, as described herein, the system 10 may be configured to operate in various modes, such as a single-cart mode, a column mode, a maintenance mode, and a depressurization mode. The cover 301 may be color coded to indicate the current type of mode for the system 10 and certain operational statuses of the system 10 in the current mode. For example, while the system 10 is operational and waiting for input in the depressurization mode, the controller 83 may control the light source 299 such that it emits a certain color, such as green. Thus, a user viewing the button 49 is aware that the system 10 is operational and may be used to sanitize a shopping cart or other object.

For illustrative purposes, assume that a user desires to operate the system 10 in the single-cart mode. To indicate such a desire, the user may depress the button 49 (e.g., press the cover 301 downward toward the housing 11) for less than five seconds. While the button 49 is depressed, the controller 83 may control the light source 299 such that it emits a certain color of light, such as blue, or a color pattern (e.g., alternating red and blue) to indicate that the pressing of the button 49 is being sensed by the system 10. When the user releases the button 49, the controller 83 may control the light source 299 to emit green again (or other color) to indicate that depressing of the button 49 is no longer sensed and the system 10 is transitioning to the desired mode of operation (i.e., single cart mode in the current example). As indicated above, in the single cart mode, the controller 83 controls the solenoid 187 such that the nozzles 86 spray sanitizing fluid for a period of time, such as four seconds, to accommodate a single shopping cart passing through the housing 11. While the nozzles 86 are spraying, the controller 83 may control the light source 299 such that it blinks (i.e., a repeating a pattern of on and off) green to indicate that the system 10 is currently operating in a mode for which spraying is activated. Once the spraying is stopped, the controller 83 may control the light source 299 to stop blinking such that it continuously emits green to again indicate that the system 10 is operational and ready to receive another input, such as another input to run signal-cart mode again or run column mode.

Now assume that a user desires to operate the system 10 in the column mode. To indicate such a desire, the user may depress the button 49 (e.g., press the cover 301 downward toward the housing 11) for more than five seconds. While the button 49 is depressed, the controller 83 may control the light source 299 such that it emits a certain color of light, such as blue, or a color pattern (e.g., alternating red and blue) to indicate that the pressing of the button 49 is being sensed by the system 10. When the user releases the button, the controller 83 may control the light source 299 to emit green again (or other color) to indicate that depressing of the button 49 is no longer sensed and the system 10 is transitioning to the desired mode of operation (i.e., column mode in the current example). As indicated above, in the column mode, the controller 83 controls the solenoid 187 such that the nozzles 86 spray sanitizing fluid for a period of time, such as thirty seconds, to accommodate a column of shopping carts passing through the housing 11. While the nozzles 86 are spraying, the controller 83 may control the light source 299 such that it blinks (i.e., a repeating a pattern of on and off) green to indicate that the system 10 is currently operating in a mode for which spraying is activated. Once the spraying is stopped, the controller 83 may control the light source 299 to stop blinking such that it continuously emits green to again indicate that the system 10 is operational and ready to receive another input, such as another input to run signal-cart mode or column mode.

In some embodiments, the controller 83 may control the light source 299 to emit a certain light color, such as red, to indicate a condition that could affect the operation of the system 10 and should be addressed by a user. As an example, when the controller 83 determines that the fluid level in the tank 63 is below a predefined threshold, the controller 83 may control the light source 299 to continuously or intermittently emit red. The controller 83 may similarly control the light source 299 to continuously or intermittently emit red in response to a detection of a leak and/or when the pump 71 has been deactivated in response to a detection of a leak or other operational fault.

Note that the hand sprayer button 100 may be similarly configured and controlled to indicate the operational state of the hand sprayer 56. In this regard, the hand sprayer button 100 is arranged to have at least one light source 319, such as one or more LEDs, as shown by FIGS. 14 and 18. In this regard, the button 100 comprises a frame 312 on which a translucent cover 311 is positioned. Such cover 311 may be spring loaded such that it moves in response to a press by a user for sensing such press and returns to its original position when the user stops pressing on the cover 311. The light source 319 is positioned within a cavity formed by the frame 312 and the cover 311 such that light from the light source 319 passes through the cover 311 and is visible to a user. Thus, the cover 301 appears to be illuminated at the color of the light from the light source 319. In some embodiments, the button 100 has multiple LEDS or other types of light sources 319 that emit different colors (e.g., red, green and blue) so that the color of the cover 311 may be changed as may be desired. As an example, as will be described in more detail below, the cover 311 may be color coded to indicate certain operational information to a user.

As an example, while the system 10 is operational and waiting for input, the controller 83 may control the light source 319 such that it emits a certain color, such as green. Thus, a user viewing the button 100 is aware that the hand sprayer 56 is operational and may be used to sanitize a shopping cart or other object.

For illustrative purposes, assume that a user desires to operate the hand sprayer 56. To indicate such a desire, the user may simultaneously depress both the start-stop button 49 and the hand sprayer button 100 for more than five seconds. Notably, requiring a user to activate simultaneously both buttons 49 and 100 for at least a threshold amount of time before activating the hand sprayer 56 for spraying makes it more difficult for an unauthorized user to successfully activate the hand sprayer 56 or determine how to activate the hand sprayer 56. While the button 49 is depressed, the controller 83 may control the light source 299 such that it emits a certain color of light, such as blue, or a color pattern (e.g., alternating red and blue) to indicate that the pressing of the button 49 is being sensed by the system 10. In addition, while the button 100 is depressed, the controller 83 may control the light source 319 such that it emits a certain color of light, such as blue, or a color pattern (e.g., alternating red and blue) to indicate that the pressing of the button 100 is being sensed by the system 10.

When the user releases the buttons 49 and 100, referred to hereafter as “the Release,” the controller 83 may control the light source 299 to emit green again (or other color) to indicate that depressing of the button 49 is no longer sensed. Upon the Release, the controller 83 may also activate the hand sprayer 56 for a period of time, referred to hereafter as a “hand sprayer cycle.” During the hand sprayer cycle, a user may use the hand sprayer 56 to spray an object to be sanitized by actuating the hand sprayer handle 57. While operating in this hand sprayer cycle, the controller 83 may also control the light source 319 to blink (i.e., a repeating a pattern of on and off) blue to indicate that the hand sprayer 56 is activated. Thus, for ten seconds after the Release, a user may cause the hand sprayer 56 to spray fluid by actuating the hand sprayer handle 57.

Note that, as described herein, the hand sprayer 56 is “activated” when (1) the controller 83 has controlled the solenoid 188 such that the ports 225 and 227 are open and the port 229 is closed and (2) the bypass valve 213 closed. Thus, when the hand sprayer 56 is activated, the tubing 207 to the bypass valve 213 and the hand sprayer 56 is pressurized so that fluid is immediately sprayed from the hand sprayer 56 when the handle 57 is actuated. At the expiration of the hand sprayer cycle following the Release, the controller 83 may deactivate the hand sprayer 56 by closing at least one of the ports 225 and 227 of the solenoid 188. If a user actuates the hand sprayer handle 57 in this state, residual fluid in the tubing 207 may be temporarily sprayed for a short time but further spraying is prevented until the hand sprayer 56 is again activated by the controller 83.

In some embodiments, the controller 83 is configured to operate the hand sprayer 56 in a state, referred to hereafter as “enabled” state, for a period of time, such as two minutes, from the Release (referred to hereafter as the “enablement period”). While the hand sprayer 56 is in the enabled state, a user may activate the hand sprayer 56 by providing an input that is different than the input described above for initiating activation of the hand sprayer (i.e., pressing of both buttons 49 and 100 for at least five seconds). Specifically, while the hand sprayer 56 is in the enabled state, a user may trigger a new hand sprayer cycle in which the hand sprayer 56 is temporarily activated for a period of time, such as ten seconds, by pressing the button 100. Thus, when the user has activated the hand sprayer 56 once, the process for reactivating the hand sprayer for a limited period of time is simplified. Once the enablement period (i.e., 2 minutes in the current example) expires, the hand sprayer 56 is deactivated and may not be used to spray fluid. However, the user may repeat the procedure described above (i.e., simultaneously depressing both buttons 49 and 100 for at least five seconds) for activating the hand sprayer 56 to begin a new enablement period.

As noted above, requiring multiple inputs, such as depressing both buttons 49 and 100, to trigger an enablement period and/or activate the hand sprayer 56 helps to prevent an unauthorized user from successfully activating the hand sprayer 56 since such process is less intuitive than simply pressing the hand sprayer button 100. However, once an authorized user has successfully activated the hand sprayer 56 or triggered an enablement period, it is likely that the authorized user is at the premises of the system 10, thereby reducing the likelihood (at least for a short period following such activation or triggering) that an unauthorized user will attempt to use the hand sprayer 56. Thus, simplifying the input required to activate the hand sprayer 56 during the enablement period reduces the burden on the user to activate the hand sprayer 56 during a time when a more complicated activation procedure is likely not needed.

Note that in some embodiments, the button 100 is not illuminated while the hand sprayer 56 is deactivated until the button 100 is pressed by a user. That is, the light source 299 is controlled so that it does not emit light until it is pressed. Not illuminating the button 100 while the hand sprayer 56 is deactivated helps to keep the button 100 from being noticed, thereby making it less likely that an unauthorized user unfamiliar with the system 10 will attempt to use the button 100. When the button 100 is pressed while in this state prior to activation of the hand sprayer 56, the button 100 may be controlled to illuminate a certain color or color pattern, such as flashing red, that suggest to an unauthorized user that pressing of the button 100 is invalid or erroneous. However, once user has provided a valid input to activate the sprayer 56, such as simultaneously pressing the buttons 49 and 100 for a predetermined amount of time (e.g., 5 seconds as described above), then the color or pattern illuminated by the button 100 may be changed to indicate that the activation input has been successfully received. As an example, the button 100 may emit a certain color, such as blue or green.

It should be emphasized that the techniques described above for activating components of the system and for indicating operational states of the system 10 are exemplary, and other techniques are possible in other embodiments. As an example, other color schemes and patterns for the buttons, as well as other types of user input or output devices, may be used. In addition, the time periods for the various modes and states described above may be varied as may be desired. Other changes and modifications would be apparent to a person of ordinary skill upon reading this disclosure.

In some embodiments, the system 10 may be configured to operate in a mode, referred to herein as “maintenance mode,” to facilitate maintenance operations, such as cleaning and/or inspecting components of the system 10, such as the strainer 166, nozzles 86, accumulator 77, and pump 71. During such mode, the tubing of the system 10 is depressurized and substantially emptied of fluid so that the tubing can be detached from the components being inspected or cleaned without spilling or spraying large volumes of sanitizing fluid. An exemplary operation and use of the system 10 in the maintenance mode will be described in more detail below.

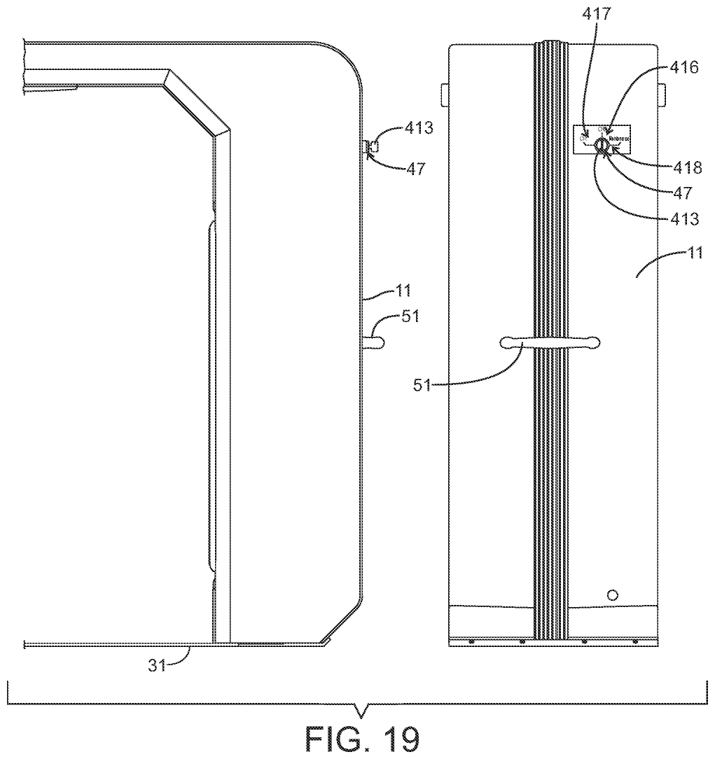

Initially, a user provides an input indicating that the system 10 is to transition to the maintenance mode. There are various types of user input devices and techniques that may be used to provide such an input. In one embodiment, the system 10 has a key switch 47 located on side of the housing 11 that can be used to indicate one or more desired modes of operation, as shown by FIG. 19. A key 413 can be inserted into the switch 47 and turned to one of three positions: an “off” position 416, an “on” position 417, and a “maintenance” position 418, and information indicative of the position of the key 413 is transmitted to the controller 83. When the key 413 is turned to the off position 416, the controller 83 deactivates the pump 71 by opening the relay 79 and controls the solenoids 187, 188 to place them in the bypass state, thereby depressurizing the tubing 206, 207. If desired, the controller 83 may power down one or more electrical components to conserve power. While the key is in the off position 416, the system 10 is not configured to spray fluid.

When the key 413 is turned to the on position 417, the controller 83 is configured to activate the pump 71 by closing the relay 79, and the system 10 may be used to spray sanitizing fluid by providing inputs via the buttons 49 and/or 100 to place the system 10 in the single-cart mode or the column mode or to activate the hand sprayer 56, as described above. When the key 413 is turned to the maintenance position 418, the controller 83 is configured to transition the system 10 to the maintenance mode.

In this regard, the controller 83 deactivates the pump 71 by opening the relay 79. Further, assuming that the system 10 is not in use for spraying (e.g., in the single-cart mode or the column mode) at this time, the solenoids 187, 188 should be in the bypass state, which is their default state unless the system 10 is in a specific mode that puts them in a different state. Thus, the tubing 206 should be depressurized and any residual fluid in the tubing 206 should have emptied through the bypass port 228 of the solenoid 187 to the tank 63.

Just prior to placing the system 10 in the maintenance mode, the user may turn the key 413 to the off position 416 and may open the access door 36. Prior to performing the work on the system 10, the system 10 may need to have the stored pressure depressurized. To do so, the user can actuate the bypass valve 213 (FIG. 14) by hand such that it is transitioned to the open state. Then, the user may turn the key 413 to the maintenance 418 to place the system in the maintenance mode in which case the controller 83 opens the solenoid 188 for a predetermined amount of time, during which the stored pressure in the accumulator 77 will be recycled or released into the tank 63. In other embodiments, the bypass valve 213 may be automatically opened by the controller 83 upon the system 10 being placed in the maintenance mode without the need of human input to the bypass valve 213.

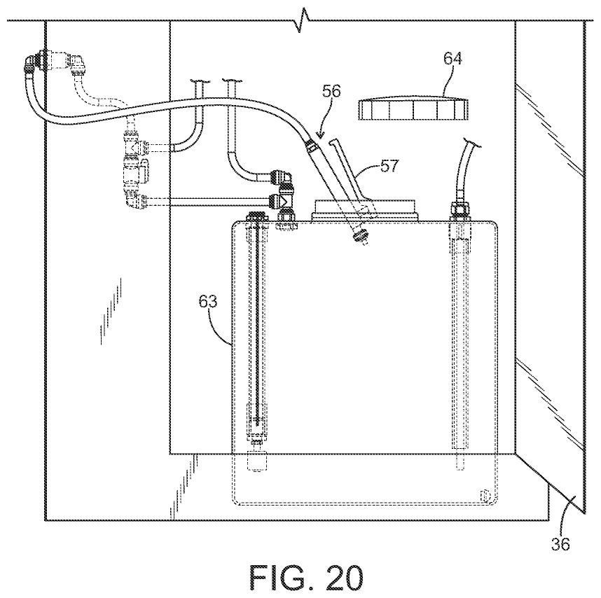

In the maintenance mode, the controller 83 may be configured to operate in the same way as described above for the column mode, except that when in the maintenance mode the pump 71 is deactivated by opening the relay 79. Thus, the controller 83 controls the solenoid 188 to close the port 229 and open the ports 225, 227, and fluid flows from the accumulator 77 through the solenoid 188 and the bypass valve 213 to the tank 63. In embodiments that do not have a bypass valve 213, the user may actuate the handle 57 of the hand sprayer 56 to start spraying fluid into the tank 63. As an example, the user may remove the lid 64 of the tank and position the tip of the sprayer 56 into the tank 63 to spray fluid into the tank 63, as shown by FIG. 20. Even though the pump 71 is deactivated, the pressure in the accumulator 77 causes fluid to be sprayed out of the hand sprayer 56. The user may repeatedly activate the hand sprayer 56 and actuate the handle 57 to spray fluid from the sprayer into the tank 63 until the accumulator 77 is depressurized and most if not all of the fluid in the system 10 and, in particular, the accumulator 77 is emptied into the tank 63. Once the accumulator 77 has been depressurized through the bypass valve 312, spraying by the hand sprayer 56, or otherwise, maintenance operations (e.g., inspecting, replacing, or cleaning components of the system 10) may be performed as desired.

Once the maintenance operations have been performed, the user may remove the handle 57 from the tank 63, replace the lid 64 on the tank 63, and close the access door 36 (if the accumulator 77 has been depressurized by spraying of fluid into the tank 63 by the hand sprayer 56. The user may also press and hold the front actuation switch 49 for a predetermined amount of time to indicate that the pump 71 is to be reactivated. In response, the controller 83 may reactivate the pump 71 for a predetermined length of time by closing the relay 79, thus allowing the user to press button 100 to open solenoid 188 for a predetermined amount of time resulting in a repressurization of the system 10. After completion of this cycle, the bypass valve 213 may be closed by the user or automatically by the controller 83 returning the system 10 to a normal mode of operation. The user may also close the access door 36 and turn the key 413 to a desired position, such as the on position 417 so that the system 10 may be used to spray sanitizing fluid or the off position 416 as may be desired. In other embodiments, other techniques for operating the system 10 in the maintenance mode are possible.

Note that maintenance operations may be further facilitated by mounting various components, such as the pump 71, the strainer 166, the accumulator 77, the solenoids 187, 188, and/or the controller 83, on a removable base 85, as described above. In this regard, if there is a failure or any other operational fault with such components, the removable base 85 may be easily removed from the system 10. If desired, the removable base 85 may be replaced with another removable base 85 having the same types of components mounted on it so that operation of the system 10 may resume while the operational fault is being diagnosed and/or corrected. As an example, a customer of the system 10 may remove the base 85 and send it, along with the components mounted thereon, to the manufacturer or other entity for diagnosing and correcting the operational fault.

FIGS. 21 and 22 depict an exemplary structure 440 on which the removable base 85 may be detachably coupled by couplers 235. The structure 440 comprises the frame 62 to which the wheels 34 are coupled. The structure 440 also comprises a vertical frame 445 that is coupled to the frame 62 by a plurality of couplers 447, such as bolts and nuts, and the removable base 85 is detachably coupled to the vertical frame 445 by the couplers 235, as described above and shown by FIG. 23.

FIG. 24 depicts the removable base 85, which in this embodiment is a sheet of high-strength metal, such as steel or aluminum. The removable base 85 has a plurality of holes 455 for receiving the couplers 235 for attachment of the base 85 to the vertical frame 445 and also couplers (not shown), such as bolts and nuts, for attachment of the components, such as the pump 71, the strainer 166, the accumulator 77, the solenoids 187, 188, and/or the controller 83, to the base 85. Note that the vertical frame 445 may have corresponding holes for receiving the couplers 235. In some embodiments, the couplers 235 may be welded or otherwise affixed to the vertical frame 445 so that the base 85 can slide off of the couplers 235 while the couplers 235 remain in a fixed position. Note that, during manufacturing, the base 85 may be cut to form a strip along an edge of the base 85, and this strip may be bent at approximately 90 degrees to form the bracket 231, as best shown by FIG. 24.

When the base 85 is to be removed, the tubing 200, 206, 207, 208 may be detached from the pump 71 and the solenoids 187, 188, and the controller 83 may be detached from the power supply 181. Further, the wing nuts 238 (FIG. 15) of the couplers 235 may be removed (e.g., unscrewed from the bolts 237 of the couplers 235), and the base 85 may then be pulled off of the bolts 237 of the couplers 235 and removed from the housing 11 through the access door 36. Another base 85 with the same types of components may then be positioned on the bolts 237, and the nuts 238 may be screwed on the bolts 237 to secure the new base 85 to the vertical frame 445. Also, the tubing 200, 206, 207, 208 may be attached to the pump 71 and the solenoids 187, 188 of the new base 85, and the controller 83 of the new base 85 may be attached to the power supply 181. At this point, the components of the new base 85 may operate, as described above, so that the system 10 is fully functional as may be desired.

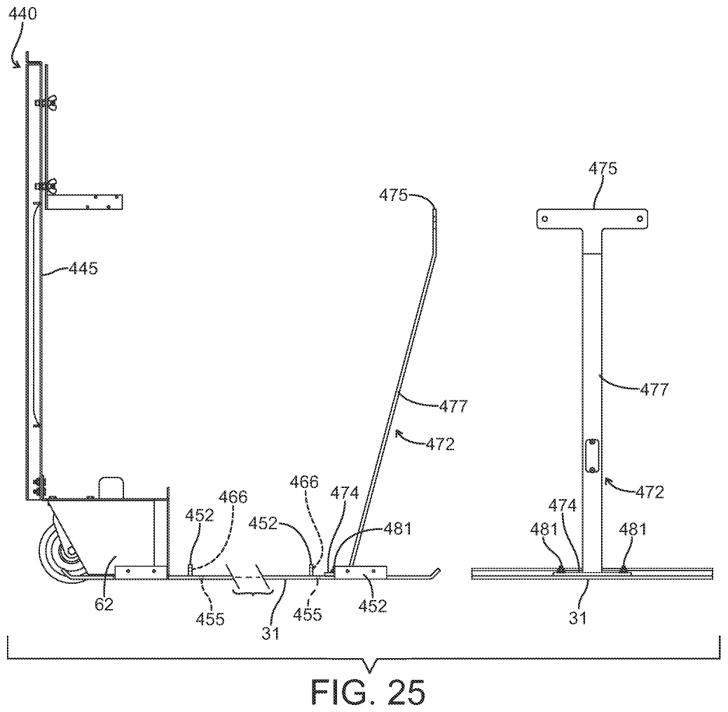

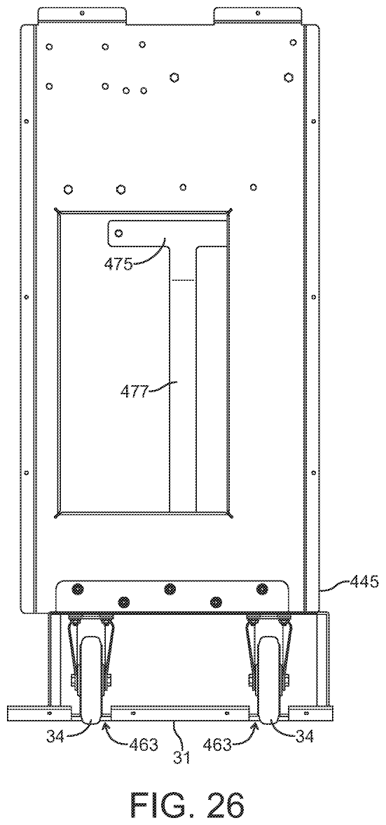

FIGS. 25-27 show an exemplary base plate 31 on which components of the system 10 including the structure 440 and housing 11 may be positioned. In one embodiment, the exemplary base plate 31 of FIGS. 25-27 is a metallic plate of about one-quarter of an inch (or a fraction thereof) in thickness of a high-strength material, such as aluminum or steel, although the base plate 31 may have other materials and dimensions in other embodiments. Further, the base plate 31 has a plurality of elongated tabs 452 extending from an upper surface of the base plate 31. In the embodiment shown by FIG. 25, each elongated tab 452 extends vertically from the plate surface and may be used to help secure the housing 11 when the housing 11 is placed on the base plate 31 during manufacturing.

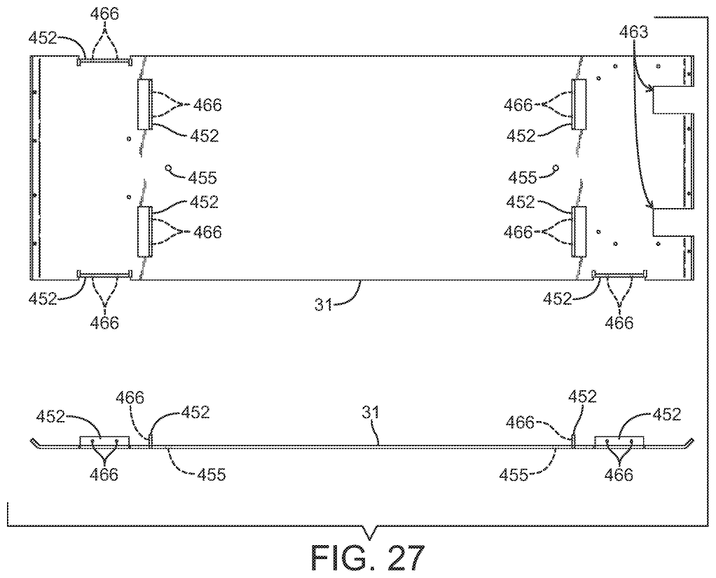

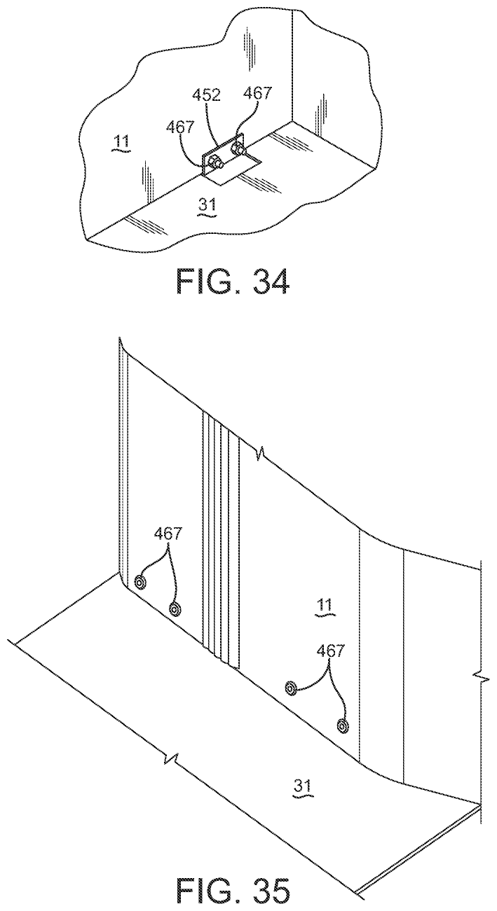

As an example, each tab 452 may have one or more holes 466, as best shown by FIG. 27, for receiving couplers (not shown in FIG. 27) for securing the housing to the tabs 452. In this regard, the tabs 452 may be positioned such that each tab 452 abuts an inner wall of the housing 11 when the housing 11 is positioned on the base plate 31, as shown by FIG. 34. In this regard, FIG. 34 shows an interior of the housing 11 after the housing 11 has been positioned on the base plate 31. Referring to FIG. 34, during manufacturing, the housing 11 is positioned on the base plate 31, as shown, such that an inner wall of the housing 11 abuts the tab 452, and couplers 467 are passed through the housing 11 and the holes 466 (FIG. 27) in the tab 452 to secure the housing to the tab 452 and thus the base plate 31. In some embodiments, the couplers 467 are pop rivets that are inserted through the housing 11 and the tab 452 by an air gun. However, other couplers are possible. As an example, each coupler 467 may instead comprise a bolt and nut arrangement having a bolt that passes through the housing and the holes 466 and a nut that can be screwed or otherwise tightened on the point to press the housing 11 and tab 452 together. Each of the other tabs 452 may be secured to the housing 11 in the same way as described above for the tab 452 shown by FIG. 34. In other embodiments, yet other techniques for securing the housing 11 to the tabs 452 are possible. FIG. 35 shows an exterior of the housing 11 depicting the couplers 467 when the couplers 467 are inserted through the housing 11 and tabs 452 for securing the housing 11 to the base plate 31.

During manufacturing, the tabs 452 may be formed by laser cutting the base plate 31 and then bending the portion of the base plate 31 along the cut lines at about 90 degrees so that the tabs 452 appear as shown in FIGS. 25-27. Further, the base plate 31 may also be laser cut during manufacturing to form grooves 463 (FIG. 27) through which the wheels 34 respectively pass to make contact with the ground or other surface on which the system 10 is positioned. In some embodiments, the base plate 31 is sandblasted and powder coated to provide enhanced grip resistance.

A plurality of anchor holes 455 (FIG. 27) are drilled into the base plate 31 along a centerline of the base plate 31. These holes 455 may be used to anchor the system 10 during shipment or use. In this regard, the system 10 may be positioned on a base or platform (not shown) having screws or bolts that pass through the holes 455 to secure the system 10 to such base or platform. If desired, the system 10 may be transported or used without securing the system 10 to a base or platform in such manner. After transport, the couplers (e.g., screws or bolts) passing through the holes 455 may be removed so that the system 10 can be removed from the base or platform used during transport and positioned as may be desired.

As shown by FIG. 25, a support structure 472 may be attached to the base plate 31 to provide mechanical support to the housing 11 and the handle 51. In some embodiments, the structure 472 is composed of a high-strength material, such as steel or aluminum, but other materials may be used if desired. As shown by FIG. 25, the structure 472 has a pair of support plates 474, 475 with an arm 477 extending from each support plate 474, 475. The structure 472 may be of unitary construction such that using couplers (not shown) to couple the arm 477 to each support plate 474, 475 is unnecessary. In this regard, the structure 472 may be formed by cutting a sheet of metal to form an outline of the arm 477 and plates 474, 475 and then bending the arm 477 at an angle (e.g., around 45 degrees or as otherwise may be desired) to each plate 474, 475.

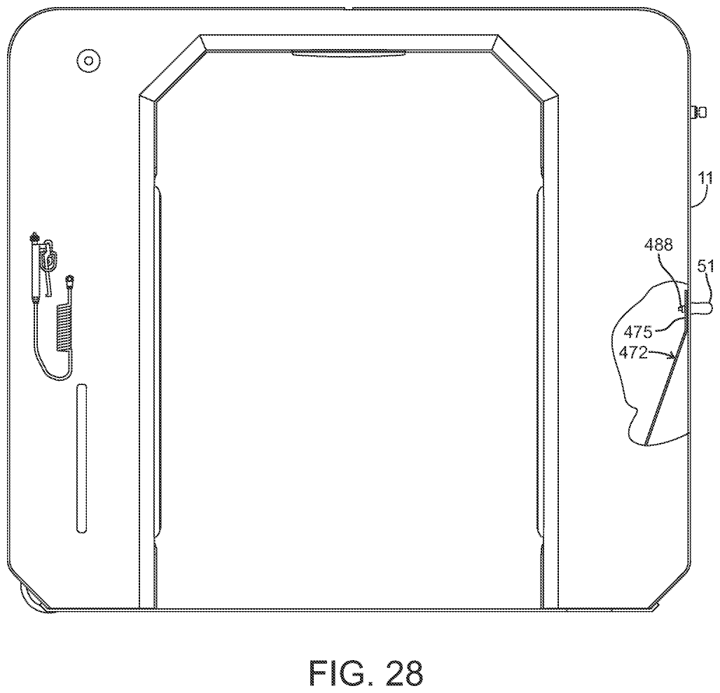

The support plate 474 is positioned flat on the upper surface of the base plate 31 and is coupled to the base plate 31 by a plurality couplers 481, such as bolts and nuts, that pass through the base plate 31 and the support plate 474. Similarly, the support plate 475 is positioned flat on an inner wall of the housing 11 and is coupled to the inner wall by a plurality of couplers 488, such as bolts and nuts, that pass through the wall of the housing 11 and the support plate 475. Notably, such couplers 488 may also pass through the handle 51 to secure the handle 51 to the exterior of the housing 11 and the support plate 475, as shown by FIG. 28. When force is applied to the handle 51 in order to move the system 10, such force is transferred to the support structure 472, which provides mechanical support to the handle 51 and transfers at least some of the load directly to the base plate 31 so that such transferred load does not need to be supported by the housing 11.

To provide additional support and stability to the housing, the structure 440 also may be coupled to the housing 11. As an example, couplers (not shown) comprising bolt and nut arrangements or other types of couplers (e.g., pop rivets) may be used to secure the housing 11 to the structure 440, such as the vertical frame 445.

After formation of the base plate 31 during manufacturing, the housing 11 may be positioned on and secured to the base plate 31 with the structures 440 and 472 located within the inner regions of the housing 11, as described above. Note that the bottoms of the legs 6, 7 of the housing 11 are both open (i.e., each leg 6, 7 has no bottom surface between the walls of the housing 11, as illustrated by FIG. 34, such that the base plate 31 is visible from inside of the housing 11). Thus, other components of the system 10 can be pre-fabricated and installed prior to the positioning of the housing 11 on the base plate 11. For example, referring to FIG. 25, the structure 440 can be positioned on the base plate 31 and other components (such as the removable base 85 (FIGS. 21 and 23), as well as the components mounted on the removable base 85, such as the pump 71, accumulator 77, controller 83, hand sprayer solenoid 188, nozzles solenoid 187, pump relay 79, and the tubing interconnecting these components) may be attached to the structure 440. In addition, the support structure 472 may be mounted on the base plate 31, as shown by FIG. 25.

After assembly of the foregoing structures and other components on the base plate 31, the housing 11 may be lowered over the aforementioned components (including the structure 440, the support structure 472, and the components mounted or otherwise positioned on the structure 440) and onto the top surface of the base plate 31. Such components may pass through the open bottom of the housing 11 as the housing 11 is being lowered to the base plate 31. The housing 11 may then be secured to the base plate 31, as described above. Note that the nozzles 86 and hand sprayer 56 may be positioned on the housing 11 prior to placement of the housing 11 on the base plate 31. Once the housing 11 is positioned on the base plate 31, the nozzles 86 may be connected to the solenoid 187 by tubing 206, and the hand sprayer 56 may be connected to the solenoid 188 by tubing 207.

Allowing pre-fabrication and installation of the structures 440, 472 and the other components mounted on those structures, such as the pump 71, accumulator 77, controller 83, hand sprayer solenoid 188, nozzles solenoid 187, and pump relay 79, facilitates manufacturing. In this regard, such structures 440, 472 and other components may be installed and positioned on the base plate 31 as appropriate and described herein without the housing 11 restricting or otherwise interfering with access to the components in any way. After installation of the structures 440, 472 and other components, the housing 11 may then be lowered over the installed structures 440, 472 and other components and positioned on the base plate 31 at which point connection of the tubing 207, 208, 209, 210, 212 to the appropriate components and securing of the housing 11 to the structures 440, 472 are the only remaining steps to complete manufacturing. Thus, much of the installation can be performed without the housing 11 interfering with the installation steps or restricting access to the structures 440, 472 and other components of the system 10.

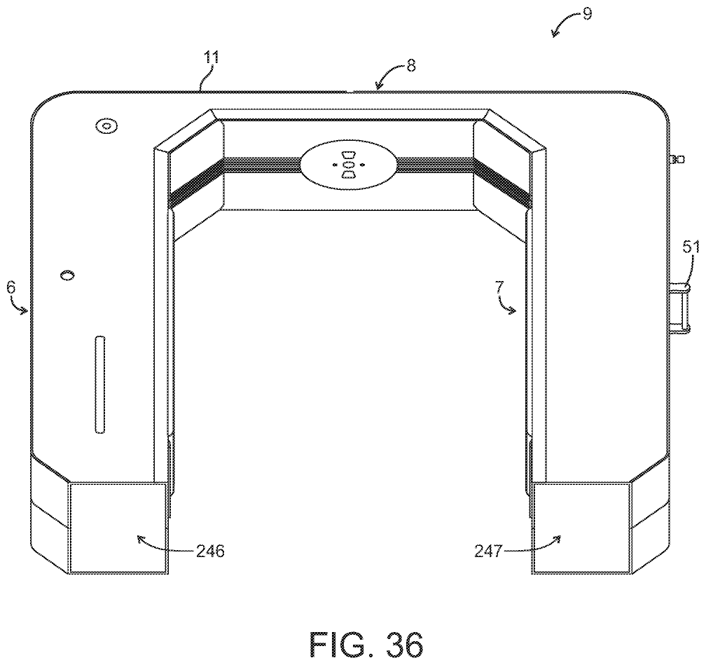

FIG. 36 depicts the housing 11 with the bottoms of the legs 6, 7 shown for illustrative purposes. As shown by FIG. 36, the bottom of the leg 6 has an opening 246 for receiving the structure 440, as well as the components mounted on the structure 440 (including at least the pump 71, the accumulator 77, the controller 83, and the solenoids 187, 188), during manufacturing as the housing 11 is being lowered onto the base plate 31. Note that any of the components described as being mounted on the structure 440 prior to lowering of the housing 11 on the base plate 31 may instead be mounted on the structure 440 after the lowering of the housing 11, if desired. The bottom of the leg 7 also has an opening 274 for receiving the structure 472 during manufacturing as the housing 11 is being lowered onto the base plate 31. If desired, the structures 440 and/or 472 may have at least some flexibility of movement so that the housing 11 may be tilted, rotated or otherwise moved relative to the components mounted on the base plate 31 as the housing 11 is being lowered to facilitate movement of the housing 11 around such components. As an example, the bolts or other couplers used to attach the structure 472 to the base plate 31 may be loosened to permit relative movement between the structure 472 and the base plate 31. Also, the structure 472 may be designed such that it can be slightly bent by hand or otherwise as the housing 11 is being lowered.

Note that the system 10 may be manufactured via other techniques in other embodiments. For example, rather than lowering the housing 11 onto the base plate 31, it is possible for the front sections 12 and the rear section 13 to be joined after the structures 440, 472 have been positioned on the base plate 31 such that it is necessary for the structures 440, 472 to pass through the openings 246, 247 as described above. Yet other variations to the manufacturing process are possible.

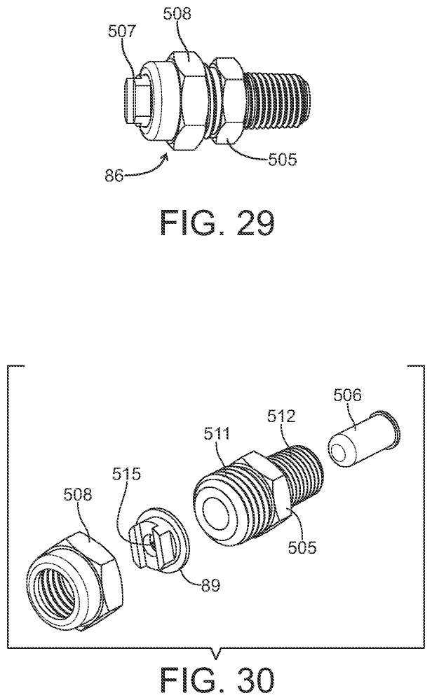

FIGS. 29 and 30 depict an exemplary embodiment of a nozzle 86. The nozzle 86 comprises a nozzle body 505, a strainer 506, a nozzle tip 89, and a ferrule screw 508. The nozzle body 505 is hollow so that fluid may pass through it. The strainer 506 is inserted into the nozzle body 505 such that the fluid flows through the strainer 506, which is composed of a mesh screen that filters particles out of the fluid as it flows. The strainer 506 also includes a check valve 152 (not shown in FIG. 30) that operates as previously described above. The nozzle tip 89 fits on an end 511 of the nozzle body 505, and the end 511 is threaded so that the ferrule screw 508 can be screwed on the end 511 to secure the nozzle tip 89. That is, the ferrule screw 508 presses against flanges of the nozzle tip 89 to hold it in place with the nozzle tip 89 extending through the screw 508, as shown by FIG. 29.

The nozzle tip 89 has a small orifice 515 from which fluid may exit to spray an object with the fluid as described above. When the nozzle 86 is mounted on the housing 11, it passes through a hole in the housing 11, and the ferrule screw 508 and tip 89 are exposed and accessible. If desired, a user may unscrew the ferrule screw 508 to remove it and the nozzle tip 89 from the nozzle body 505, which may be attached to the housing 11, as will be described in more detail below. Thus, the user may remove and clean the nozzle tip 89 without having to open an access door 36-38 or other compartment of the housing 11 or to access the inner region of the housing 11, thereby facilitating cleaning of the nozzle tips 89.

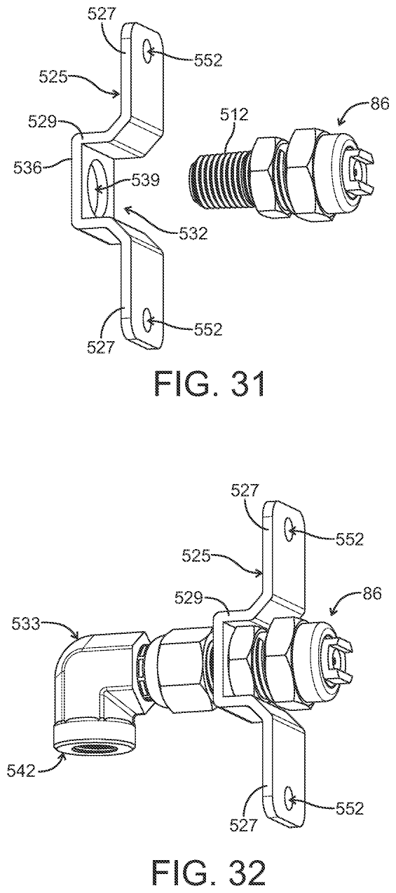

FIG. 31 depicts an exemplary bracket 525 that may be used to mount nozzles 86 on the housing 11. The exemplary bracket 525 of FIG. 31 has a pair of flat flanges 527 that are joined by a raised portion 529 that forms an A-frame. The raised portion 529 defines a cavity 532 in which the portions of the nozzle 86 may be situated when the bracket 525 is used to secure the nozzle 86, as shown by FIG. 32. The raised portion 529 has a flat surface 536 at the top of the A-frame, and this flat surface 536 has a hole 539 through which a male end 512 of the nozzle body 505 (FIG. 29) passes. The male end 512 of the nozzle body 505 is threaded such that it can be attached to a swivel fitting 533 by screwing a threaded female end of the fitting 533 onto the end 512. Tubing 206 may be attached to an opposite end 542 of the swivel fitting 533. The swivel fitting 533 is hollow such that fluid from the tubing 206 passes through the fitting 533 to the nozzle 86, which sprays the fluid from the nozzle tip 89 (FIG. 30). As shown by FIGS. 31 and 32, each flange 527 has a hole 552 through which a coupler (not shown in FIGS. 30 and 31), such as screw or bolt, may pass to secure the bracket 525 to the housing 11.

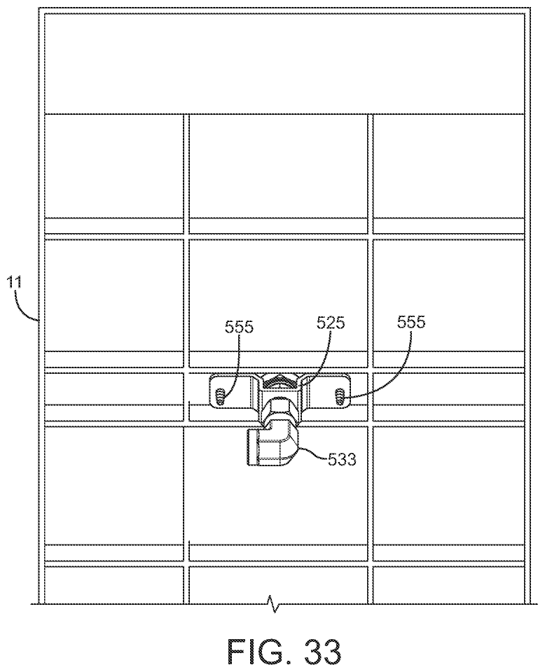

In this regard, FIG. 33 shows the bracket 525 attached to the housing 11. As shown by FIG. 33, a screw 555 passes through each hole 552 (FIG. 32) in a respective flange 527 in order to secure the bracket 525 to an inner wall of the housing 11. As noted above, when the bracket 525 is mounted to the housing 11, as shown by FIG. 33, the inner wall of the housing 11 has a hole (not shown in FIG. 32) through which the nozzle 86 passes so that the nozzle tip 89 and ferrule screw 508 (FIG. 30) are exposed and accessible from outside the housing 11.

While a sanitization system has been shown in various forms and configurations, it will be obvious to those skilled in the art that it is not so limited but is susceptible of various changes and modifications without departing from the spirit thereof. For example, the system may be used for articles different than shopping carts, baskets, and other items described herein. Further, the controller 83 can be adjusted to control the functioning (e.g., timing) of spraying by the nozzles and the positioning of the nozzles can be easily altered to suit different sizes of articles.

Now, therefore, the following is claimed:

1. A method for manufacturing a sanitization system, comprising:
positioning a structure on a base plate;
coupling a controller, a pump, and an accumulator to a base;
coupling the pump to the accumulator with first tubing;
coupling the base to the structure such that the base is removable from the structure;
positioning a housing forming an arch on the base plate such that the structure is within an inner region of the housing;
coupling, with second tubing, the accumulator to a plurality of nozzles mounted on the housing;
coupling, with third tubing, the pump to a tank for holding a sanitization fluid; and
securing the housing to the base plate.

2. The method of claim 1, further comprising:
coupling the base plate to a first end of an arm; and
coupling a second end of the arm to the housing and a handle on an exterior of the housing, wherein the arm is in an inner region of the housing.

3. The method of claim 1, further comprising coupling at least one solenoid to the structure, wherein the coupling the accumulator comprises:
coupling at least one of the nozzles to the at least one solenoid with the second tubing; and
coupling the at least one solenoid to the accumulator with the second tubing.

4. The method of claim 3, further comprising coupling the at least one solenoid to the tank with fourth tubing.

5. The method of claim 1, wherein the coupling the controller, the pump, and the accumulator to the base is performed before the coupling the base to the structure.

6. The method of claim 1, wherein the arch has at least a first leg and a second leg, wherein a bottom of the first leg has a first opening, and wherein the positioning the housing comprises moving the housing relative to the structure while the structure is positioned on the base plate such that the first leg of the arch receives the structure through the first opening.

7. The method of claim 6, further comprising:
coupling the base plate to a first end of an arm; and
coupling a second end of the arm to the housing and a handle on an exterior of the housing, wherein the arm is in an inner region of the housing.

8. The method of claim 7, wherein a bottom of the second leg has a second opening, and wherein the positioning the housing comprises moving the housing relative to the arm while the arm is coupled to the base plate such that the second leg of the arch receives the arm through the second opening.

9. A method for manufacturing a sanitization system, comprising:
positioning a structure on a base plate;
coupling a controller, a pump, and an accumulator to the structure;
coupling the pump to the accumulator with first tubing;
positioning a housing forming an arch on the base plate while the structure is positioned on the base plate such that the structure is within an inner region of the housing, wherein the arch has at least a first leg and a second leg, wherein a bottom of the first leg has a first opening, and wherein the positioning the housing comprises moving the housing relative to the structure such that the first leg of the arch receives the structure through the first opening;
coupling, with second tubing, the accumulator to a plurality of nozzles mounted on the housing;
coupling, with third tubing, the pump to a tank for holding a sanitization fluid; and
securing the housing to the base plate.

10. The method of claim 9, wherein the moving is performed such that the first leg of the arch receives the controller, the pump, and the accumulator through the first opening.

11. The method of claim 9, further comprising:
coupling the base plate to a first end of an arm; and
coupling a second end of the arm to the housing and a handle on an exterior of the housing, wherein the arm is in an inner region of the housing.

12. The method of claim 11, wherein a bottom of the second leg has a second opening, and wherein the positioning the housing comprises moving the housing relative to the arm while the arm is coupled to the base plate such that the second leg of the arch receives the arm through the second opening.

13. The method of claim 9, further comprising coupling at least one solenoid to the structure, wherein the coupling the accumulator comprises:
coupling at least one of the nozzles to the at least one solenoid with the second tubing; and
coupling the at least one solenoid to the accumulator with the second tubing.

14. The method of claim 13, further comprising coupling the at least one solenoid to the tank with fourth tubing.

15. The method of claim 9, wherein the coupling the controller, the pump, and the accumulator to the structure is performed before the positioning the housing.
